(19) United States
(12) Patent Application Publication
(10) Pub. No.: US 2007/0141627 A1
(43) Pub. Date: Jun. 21, 2007

(54) Systemic Lupus Erythematosus

(76) Inventors: Timothy W. Behrens, Burlingame, CA (US); Emily C. Gillespie, Savage, MN (US); Peter K. Gregersen, Larchmont, NY (US); Jason W. Bauer, St. Paul, MN (US)

Correspondence Address: FISH & RICHARDSON P.C., PO BOX 1022, MINNEAPOLIS, MN, 55440-1022, US

(21) Appl. No.: 11/550,673

(22) Filed: Oct. 18, 2006

Related U.S. Application Data
(60) Provisional application No. 60/728,617, filed on Oct. 19, 2005.

(51) Int. Cl. G01N 33/53

(52) U.S. Cl. 435/7.1

(57) ABSTRACT

This document relates to methods and materials involved in diagnosing SLE. For example, this document provides arrays for detecting polypeptides that can be used to diagnose SLE in a mammal. In addition, methods and materials for assessing SLE activity, determining the likelihood of experiencing active SLE, and detecting SLE treatment effectiveness are provided herein.

### CROSS-REFERENCE TO RELATED APPLICATIONS

This application claims the benefit of priority to U.S. Provisional Application Ser. No. 60/728,617, filed on Oct. 19, 2005.

### STATEMENT AS TO FEDERALLY SPONSORED RESEARCH

Funding for the work described herein was provided in part by the National Institute of Arthritis and Musculoskeletal Diseases (grant no. NIH N01-AR-1-2256).

### BACKGROUND

1. Technical Field

This document relates to methods and materials involved in diagnosing systemic lupus erythematosus (SLE). For example, this document relates to methods and materials involved in diagnosing SLE, assessing a mammal's susceptibility to develop SLE, and assessing SLE activity.

2. Background Information

SLE is a chronic, inflammatory autoimmune disease characterized by the production of autoantibodies having specificity for a wide range of self-antigens. SLE autoantibodies mediate organ damage by directly binding to host tissues and by forming immune complexes that deposit in vascular tissues and activate immune cells. Organs targeted in SLE include the skin, kidneys, vasculature, joints, various blood elements, and the central nervous system (CNS). The severity of disease, the spectrum of clinical involvement, and the response to therapy vary widely among patients. This clinical heterogeneity makes it challenging to diagnose and manage lupus.

### SUMMARY

This document relates to methods and materials involved in diagnosing SLE. For example, this document relates to methods and materials involved in diagnosing SLE, assessing a mammal's susceptibility to develop SLE, and assessing SLE activity. For example, this document provides arrays for detecting polypeptides that can be used to diagnose SLE in a mammal. Such arrays can allow clinicians to diagnose SLE based on a determination of the levels of many polypeptides that are differentially regulated in SLE patients as compared to healthy controls. This document also provides methods and materials that can be used to assess SLE activity. Assessing SLE activity can allow clinicians to identify patients with active SLE. In addition, this document provides methods and materials that can be used to assess the likelihood that a patient will experience active SLE. For example, a patient found to have serum containing one or more polypeptides listed in Table 3 at a level that is greater than or less than the average level observed in control serum can be classified as being likely to experience active SLE. This document also provides methods and materials that can be used to determine whether or not a mammal responds to an SLE treatment. For example, patients receiving an SLE treatment (e.g., an anti-IFN treatment) who are found have serum that no longer contains one or more IFN-regulated chemokines at a level greater than or less than the average level observed in control serum can be classified as responding to that SLE treatment.

Typically, a diagnosis of SLE can be made on the basis of 11 criteria defined by the American College of Rheumatology (ACR). These criteria include malar rash, discoid rash, photosensitivity, oral ulcers, arthritis, serositis, renal disorder, neurologic disorder, hematologic disorder, immunologic disorder, and antinuclear antibody (Tan et al. (1982) Arthritis Rheum 25:1271-1277). A mammal (e.g., a human) can be clinically diagnosed with SLE if he or she meets at least four of the eleven criteria.

This document is based, in part, on the discovery of polypeptides that are differentially regulated between SLE patients and healthy controls. This document also is based, in part, on the discovery that the serum levels of polypeptides can be used to distinguish mammals with SLE from healthy mammals. For example, the serum levels for the polypeptides listed in Table 2 can be assessed to diagnose SLE.

For the purpose of this document, the term “activity signature 1” as used herein refers to a serum polypeptide profile where one or more (e.g., two, three, four, five, six, seven, eight nine, ten, 15, 20, 25, 30, 35, 40, 45, 50, 55, 60, 65, or more) of the polypeptides listed in Table 2 are present at a level greater than or less than the level observed in control serum from a control mammal. In some cases, the activity signature 1 can be a serum polypeptide profile where 10, 20, 30, 40, 50, 60, 70, 80, 90, or 100 percent of the polypeptides listed in Table 2 are present at a level greater than or less than the level observed in control serum from a control mammal. The term “activity signature 2” as used herein refers to a serum polypeptide profile where one or more (e.g., two, three, four, five, six, seven, eight nine, ten, 15, 20, 25, 27, or more) of the polypeptides listed in Table 3 are present at a level greater than or less than the level observed in control serum from a control mammal. In some cases, the activity signature 2 can be a serum polypeptide profile where 10, 20, 30, 40, 50, 60, 70, 80, 90, or 100 percent of the polypeptides listed in Table 3 are present at a level greater than or less than the level observed in control serum from a control mammal. The term “activity signature 3” as used herein refers to a serum polypeptide profile where one or more (e.g., two, three, four, five, six, seven, eight nine, ten, 15, 18, or more) of the polypeptides listed in Table 4 are present at a level greater than or less than the level observed in control serum from a control mammal. In some cases, the activity signature 3 can be a serum polypeptide profile where 10, 20, 30, 40, 50, 60, 70, 80, 90, or 100 percent of the polypeptides listed in Table 4 are present at a level greater than or less than the level observed in control serum from a control mammal. The term “activity signature 4” as used herein refers to a serum polypeptide profile where one or more (e.g., two, three, four, five, six, or more) polypeptides selected from the group consisting of CCL2, CCL3, CCL8, CCL19, CXCL9, CXCL10, and CXCL11 polypeptides are present at a level greater than or less than the level observed in control serum from a control mammal. In some cases, the activity signature 4 can be a serum polypeptide profile where 10, 20, 30, 40, 50, 60, 70, 80, 90, or 100 percent of the polypeptides selected from the group consisting of CCL2, CCL3, CCL8, CCL19, CXCL9, CXCL10, and CXCL11polypeptides are present at a level greater than or less than the level observed in control serum from a control mammal.

In general, one aspect of this document features a method for identifying a mammal having systemic lupus erythematosus. The method comprises, or consists essentially of, (a) determining whether or not serum from a mammal comprises a signature selected from the group consisting of an activity signature 1, an activity signature 2, an activity signature 3, and an activity signature 4, and (b) classifying the mammal as having systemic lupus erythematosus if the serum comprises the signature and classifying the mammal as not having systemic lupus erythematosus if the serum does not comprise the signature. The mammal can be a human. The method can include determining whether or not the serum comprises the activity signature 1. The method can include determining whether or not the serum comprises the activity signature 2. The method can include determining whether or not the serum comprises the activity signature 3. The method can include determining whether or not the serum comprises the activity signature 4.

In another embodiment, this document features a method for identifying a mammal having systemic lupus erythematosus. The method comprises, or consists essentially of, (a) determining whether or not serum from a mammal contains one or more of the polypeptides listed in Table 2, 3, or 4 at a level that is greater than or less than the average level of the same one or more polypeptides observed in control serum obtained from one or more control mammals, and (b) classifying the mammal as having systemic lupus erythematosus if the serum contains the one or more polypeptides and classifying the mammal as not having systemic lupus erythematosus if the serum does not contain the one or more polypeptides. The mammal can be a human. The method can include determining whether or not the serum contains one or more polypeptides (e.g., 2, 3, 4, 5, 6, 7, 8, 9, or 10 polypeptides) listed in Table 2. The method can include determining whether or not the serum contains one or more polypeptides (e.g., 2, 3, 4, 5, 6, 7, 8, 9, or 10 polypeptides) listed in Table 3. The method can include determining whether or not the serum contains one or more polypeptides (e.g., 2, 3, 4, 5, 6, 7, 8, 9, or 10 polypeptides) listed in Table 4.

In another embodiment, this document features a method for identifying a mammal having systemic lupus erythematosus. The method comprises, or consists essentially of, (a) determining whether or not serum from a mammal contains one or more of the polypeptides selected from the group consisting of CCL2, CCL3, CCL8, CCL19, CXCL9, CXCL10, and CXCL11 polypeptides at a level that is greater than or less than the average level of the same one or more polypeptides observed in control serum obtained from one or more control mammals, and (b) classifying the mammal as having systemic lupus erythematosus if the serum contains the one or more polypeptides and classifying the mammal as not having systemic lupus erythematosus if the serum does not contain the one or more polypeptides. The mammal can be a human.

In another embodiment, this document features a method for assessing systemic lupus erythematosus disease activity. The method comprises, or consists essentially of, (a) determining whether or not serum from a mammal comprises a signature selected from the group consisting of an activity signature 1, an activity signature 2, an activity signature 3, and an activity signature 4, and (b) classifying the mammal as having active systemic lupus erythematosus disease if the serum comprises the signature and classifying the mammal as not having active systemic lupus erythematosus disease if the serum does not comprise the signature. The mammal can be a human. The method can include determining whether or not the serum comprises the activity signature 1. The method can include determining whether or not the serum comprises the activity signature 2. The method can include determining whether or not the serum comprises the activity signature 3. The method can include determining whether or not the serum comprises the activity signature 4.

In another embodiment, this document features a method for assessing systemic lupus erythematosus disease activity. The method comprises, or consists essentially of, (a) determining whether or not serum from a mammal contains one or more of the polypeptides listed in Table 2, 3, or 4 at a level that is greater than or less than the average level of the same one or more polypeptides observed in control serum obtained from one or more control mammals, and (b) classifying the mammal as having active systemic lupus erythematosus disease if the serum contains the one or more polypeptides and classifying the mammal as not having active systemic lupus erythematosus disease if the serum does not contain the one or more polypeptides. The mammal can be a human. The method can include determining whether or not the serum contains one or more polypeptides (e.g., 2, 3, 4, 5, 6, 7, 8, 9, or 10 polypeptides) listed in Table 2. The method can include determining whether or not the serum contains one or more polypeptides (e.g., 2, 3, 4, 5, 6, 7, 8, 9, or 10 polypeptides) listed in Table 3. The method can include determining whether or not the serum contains one or more polypeptides (e.g., 2, 3, 4, 5, 6, 7, 8, 9, or 10 polypeptides) listed in Table 4.

In another embodiment, this document features a method for assessing systemic lupus erythematosus disease activity. The method comprises, or consists essentially of, (a) determining whether or not serum from a mammal contains one or more of the potypeptides selected from the group consisting of CCL2, CCL3, CCL8, CCL19, CXCL9, CXCL10, and CXCL11 polypeptides, and (b) classifying the mammal as having active systemic lupus erythematosus disease if the serum contains the one or more polypeptides and classifying the mammal as not having active systemic lupus erythematosus disease if the serum does not contain the one or more polypeptides. The mammal can be a human.

In another embodiment, this document features a method for determining whether or not a mammal is likely to experience active systemic lupus erythematosus disease. The method comprises, or consists essentially of, (a) determining whether or not serum from a mammal comprises a signature selected from the group consisting of an activity signature 1, an activity signature 2, an activity signature 3, and an activity signature 4, and (b) classifying the mammal as being likely to experience the active systemic lupus erythematosus disease if the serum comprises the signature and classifying the mammal as not being likely to experience the active systemic lupus erythematosus disease if the serum does not comprise the signature. The mammal can be a human. The method can include determining whether or not the serum comprises the activity signature 1. The method can include determining whether or not the serum comprises the activity signature 2. The method can include determining whether or not the serum comprises the activity signature 3. The method can include determining whether or not the serum comprises the activity signature 4.

In another embodiment, this document features a method for determining whether or not a mammal is likely to experience active systemic lupus erythematosus disease. The method comprises, or consists essentially of, (a) determining whether or not serum from a mammal contains one or more of the polypeptides listed in Table 2, 3, or 4 at a level that is greater than or less than the average level of the same one or more polypeptides observed in control serum obtained from one or more control mammals, and (b) classifying the mammal as being likely to experience the active systemic lupus erythematosus disease if the serum contains the one or more polypeptides and classifying the mammal as not being likely to experience the active systemic lupus erythematosus disease if the serum does not contain the one or more polypeptides. The mammal can be a human. The method can include determining whether or not the serum contains one or more polypeptides (e.g., 2, 3, 4, 5, 6, 7, 8, 9, or 10 polypeptides)listed in Table 2. The method can include determining whether or not the serum contains one or more polypeptides (e.g., 2, 3, 4, 5, 6, 7, 8, 9, or 10 polypeptides) listed in Table 3. The method can include determining whether or not the serum contains one or more polypeptides (e.g., 2, 3, 4, 5, 6, 7, 8, 9, or 10 polypeptides) listed in Table 4.

In another embodiment, this document features a method for determining whether or not a mammal is likely to experience active systemic lupus erythematosus disease. The method comprises, or consists essentially of, (a) determining whether or not serum from a mammal contains one or more of the polypeptides selected from the group consisting of CCL2, CCL3, CCL8, CCL19, CXCL9, CXCL10, and CXCL11 polypeptides at a level that is greater than or less than the average level of the same one or more polypeptides observed in control serum obtained from one or more control mammals, and (b) classifying the mammal as being likely to experience the active systemic lupus erythematosus disease if the serum contains the one or more polypeptides and classifying the mammal as not being likely to experience the active systemic lupus erythematosus disease if the serum does not contain the one or more polypeptides. The mammal can be a human.

In another embodiment, this document features a method for assessing effectiveness of a treatment for systemic lupus erythematosus disease. The method comprises, or consists essentially of, determining whether or not a mammal having systemic lupus erythematosus disease and having received a treatment for the systemic erythematosus disease comprises serum having a signature selected from the group consisting of an activity signature 1, an activity signature 2, an activity signature 3, and an activity signature 4 at a level less than the level present in an earlier obtained serum sample from the mammal, where the presence of the serum indicates that the treatment is effective. The mammal can be a human. The method can include determining whether or not the serum comprises the activity signature 1. The method can include determining whether or not the serum comprises the activity signature 2. The method can include determining whether or not the serum comprises the activity signature 3. The method can include determining whether or not the serum comprises the activity signature 4.

In another embodiment, this document features a method for assessing effectiveness of a treatment for systemic lupus erythematosus disease. The method comprises, or consists essentially of, determining whether or not a mammal having systemic lupus crythematosus disease and having received a treatment for the systemic erythematosus disease has serum containing one or more of the polypeptides listed in Table 2, 3, or 4 at a level less than the level present in an earlier obtained serum sample from the mammal, where the presence of the serum indicates that the treatment is effective. The mammal can be a human. The method can include determining whether or not the serum contains one or more of the polypeptides (e.g., 2, 3, 4, 5, 6, 7, 8, 9, or 10 polypeptides) listed in Table 2. The method can include determining whether or not the serum contains one or more of the polypeptides (e.g., 2, 3, 4, 5, 6, 7, 8, 9, or 10 polypeptides) listed in Table 3. The method can include determining whether or not the serum contains one or more of the polypeptides (e.g., 2, 3, 4, 5, 6, 7, 8, 9, or 10 polypeptides) listed in Table 4.

In yet another aspect, this document features a method for assessing effectiveness of a treatment for systemic lupus erythematosus disease. The method comprises, or consists essentially of, determining whether or not a mammal having systemic lupus erythematosus disease and having received a treatment for the systemic erythematosus disease has serum containing one or more of the polypeptides selected from the group consisting of CCL2, CCL3, CCL8, CCL19, CXCL9, CXCL10, and CXCL11 polypeptides at a level less than the level present in an earlier obtained serum sample from the mammal, where the presence of the serum indicates that the treatment is effective. The mammal can be a human.

In yet another aspect, this document features an array for detecting polypeptides. The array comprises, or consists essentially of, at least 5 polypeptides (e.g., at least 6, 7, 8, 9, 10, 15, 20, 30, or more polypeptides) capable of detecting polypeptides, wherein each of the at least 5 polypeptides has a different amino acid sequence. The array can contain at least 50 polypeptides capable of detecting polypeptides, wherein each of the at least 50 polypeptides has a different amino acid sequence. The polypeptides capable of detecting polypeptides can be antibodies or antibody fragments. An antibody or antibody fragment can be capable of detecting (e.g., via binding at typical antibody affinity) a polypeptide listed in Table 2, 3, or 4. The array can contain glass.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting IFN gene expression scores for IFN-hi SLE, IFN-lo SLE and control subjects. Whole blood gene expression microarrays were used to identify 82 type I IFN-regulated genes that distinguished 81 SLE cases from 42 controls. These genes were used to derive a normalized IFN gene score. Plotted are IFN gene scores for 15 IFN-hi SLE (mean±SD, 41.0±4.8), 15 IFN-lo SLE (14.4±2.8), and 15 controls (12.1±1.9).

FIG. 2 is a set of graphs plotting serum levels of CCL2 (upper panel) or CXCL9 (lower panel) measured using Luminex bead-based immunoassays against the levels measured using antibody arrays. Linear regression analysis was performed. The units are pg/mL.

FIG. 3 is a graph plotting correlation coefficients between serum polypeptide levels and blood gene expression levels. Linear regression analysis was used to determine the correlations between serum polypeptide levels and gene expression levels in whole blood measured concurrently using Affymetrix microarrays. P<0.05 thresholds are shown by dotted lines.

FIG. 4 is a graph plotting chemokine polypeptide scores for 15 IFN-hi SLE (mean±SD=2.8±1.2), 15 IFN-lo SLE(1.4±0.5) and 15 controls (1.0±0.2).

### DETAILED DESCRIPTION

This document provides methods and materials involved in diagnosing SLE, such as methods and materials involved in diagnosing SLE and assessing a mammal's susceptibility to develop SLE. For example, this document provides arrays for detecting polypeptides that can be used to diagnose SLE in a mammal. Such arrays can allow clinicians to diagnose SLE based on a determination of, for example, serum levels of many polypeptides that are differentially expressed. In addition, the methods and materials provided herein can be used to assess SLE activity, determine the likelihood of experiencing active SLE, and detect SLE treatment effectiveness.

As described herein, this document provides methods for diagnosing a mammal (e.g., a human) as having SLE. In some embodiments, a mammal can be diagnosed as having SLE if it is determined that a sample from the mammal (e.g., a urine or serum sample) contains one or more of the polypeptides listed in Table 2, 3, or 4 at a level that is greater than or less than the average level of the same one or more polypeptides observed in control sample obtained from control mammals. In some cases, a mammal can be diagnosed as having SLE if it is determined that a sample from the mammal (e.g., the mammal's serum) contains one or more of the polypeptides selected from the group consisting of CCL2, CCL3, CCL8, CCL19, CXCL9, CXCL10, and CXCL11 polypeptides at a level that is greater than the average level of the same one or more polypeptides observed in control sample (e.g., control serum) obtained from control mammals.

The mammal can be any mammal such as a human, dog, mouse, or rat. Any method can be used to obtain serum for evaluation. For example, a sample such as serum can be obtained by peripheral venipuncture and evaluated to determine if it contains (1) one or more of the polypeptides listed in Table 2 at a level that is greater than or less than the average level observed in control serum, (2) one or more of the polypeptides listed in Table 3 at a level that is greater than or less than the average level observed in control serum, (3) one or more of the polypeptides listed in Table 4 at a level that is greater than or less than the average level observed in control serum, or (4) one or more of the polypeptides selected from the group consisting of CCL2, CCL3, CCL8, CCL19, CXCL9, CXCL10, and CXCL11 polypeptides at a level that is greater than the average level observed in control serum. The level of any number of polypeptides listed in Table 2, 3, or 4 can be evaluated to diagnose SLE. For example, the level of one or more than one (e.g., two, three, four, five, six, seven, eight, nine, ten, 15, 20, 25, 30, or more than 30) of the polypeptides listed in Table 2, 3, or 4 can be used. Examples of polypeptide combinations that can be used include, without limitation, CCL19 and CXCL9 polypeptides; ACE, IP10, IL6, and MMP7 polypeptides; IL6, IL15, IL18, IL2SRA, and MMP7 polypeptides; and CCL2, CCL3, CCL8, CCL19, CXCL9, CXCL10, and CXCL11 polypeptides. It will be appreciated that urine samples can be used in place of serum samples for the methods and materials described herein and that urine samples can be obtained using standard urine collection techniques. In some cases, both serum and urine samples can be used as described herein.

The serum level can be greater than or less than the average level observed in control serum obtained from control mammals. Typically, a polypeptide can be classified as being present at a level that is greater than or less than the average level observed in control serum if the levels differ by at least 10, 20, 30, 40, 50, 60, 70, 80, 90, or more percent. In some cases, a polypeptide can be classified as being present at a level that is greater than or less than the average level observed in control serum if the levels differ by greater than 1-fold (e.g., 1.5-fold, 2-fold, 3-fold, or more than 3-fold). Control serum typically is of the same species as the mammal being evaluated. In some cases, control serum can be obtained from one or more mammals that are from the same species as the mammal being evaluated. When diagnosing SLE, control serum can be isolated from healthy mammals such as healthy humans who do not have SLE. Any number of control mammals can be used to obtain the control serum. For example, control serum can be obtained from one or more healthy mammals (e.g., at least 5, at least 10, at least 15, at least 20, or more than 20 control mammals).

Any method can be used to determine whether or not a polypeptide is present at a level that is greater than or less than the average level observed in control serum. For example, the level of a particular polypeptide can be measured using, without limitation, immuno-based assays (e.g., ELISA), western blotting, arrays for detecting polypeptides, two-dimensional gel analysis, chromatographic separation, or mass spectroscopy. Methods of using arrays for detecting polypeptides include, without limitation, those described herein. Such methods can be used to determine simultaneously the relative levels of multiple polypeptides.

This document also provides methods and materials for diagnosing a mammal (e.g., a human) as having SLE disease activity. A number of measures can typically be used to define active SLE disease. Such disease activity measures include, without limitation, the SLE Disease Activity Index (SLEDAI), the Systemic Lupus Activity Measure (SLAM), a physicians global assessment (PGA), the erythrocyte sedimentation rate (ESR), the titers of anti-dsDNA antibodies, the white blood cell (WBC) count, and the hematocrit. A mammal can be diagnosed as having active or inactive SLE disease based on one or more disease activity measures. For example, a human having a PGA≧1.5 and SLEDAI≧3 can be diagnosed as having active SLE disease. In some cases, a human having a PGA≦1 and SLEDAI≦2 can be diagnosed as having inactive SLE disease.

In some embodiments, a mammal can be diagnosed as having active SLE disease if it is determined that the mammal's serum contains one or more of the polypeptides listed in Table 2, 3, or 4 at a level that is greater than or less than the average level of the same one or more polypeptides observed in control serum obtained from control mammals. In some cases, a mammal can be diagnosed as having active SLE disease if it is determined that the mammal's serum contains one or more of the polypeptides selected from the group consisting of CCL2, CCL3, CCL8, CCL19, CXCL9, CXCL10, and CXCL11 polypeptides at a level that is greater than the average level of the same one or more polypeptides observed in control serum obtained from control mammals.

Once a mammal (e.g., a human) has been diagnosed as having active SLE disease, the mammal can be subsequently evaluated or monitored over time for an increase or a decrease in SLE disease activity. For example, a mammal can be assessed as having an increased or decreased SLE disease activity if it is determined that the mammal's serum contains one or more polypeptides listed in Table 2, 3, or 4 at a level that is greater than or less than the average level of the same one or more polypeptides observed in serum obtained previously from the same mammal. In some cases, a mammal can be assessed as having an increased or decreased SLE disease activity if it is determined that the mammal's serum contains one or more polypeptides selected from the group consisting of CCL2, CCL3, CCL8, CCL19, CXCL9, CXCL10, and CXCL11 polypeptides at a level that is greater than or less than the average level of the same one or more polypeptides observed in serum obtained previously from the same mammal. For example, a mammal can be assessed as having an increased SLE disease activity if it is determined that the mammal's serum contains one or more polypeptides selected from the group consisting of CCL2, CCL3, CCL8, CCL19, CXCL9, CXCL10, and CXCL11 polypeptides at a level that is greater than the average level of the same one or more polypeptides observed in serum obtained previously from the same mammal. In some cases, a mammal can be assessed as having a decreased SLE disease activity if it is determined that the mammal's serum contains one or more polypeptides selected from the group consisting of CCL2, CCL3, CCL8, CCL19, CXCL9, CXCL10, and CXCL11 polypeptides at a level that is less than the average level of the same one or more polypeptides observed in serum obtained previously from the same mammal. A mammal can be monitored for SLE disease activity over any period of time with any frequency. For example, a mammal can be monitored every three months for one year or once a year for as long as the mammal is alive. In some cases, the SLE disease activity of a mammal can be monitored with a single follow-up assessment.

A mammal can also be assessed for SLE disease activity before, during, and after being treated for SLE. For example, a mammal can be assessed for SLE disease activity while being treated with anti-interferon therapy, hydroxychloroquinone, steroids, or immunosuppressive drugs. Assessing a mammal for SLE disease activity during treatment of the mammal for SLE can allow the effectiveness of the SLE therapy to be determined. For example, a decrease in SLE activity during or after treatment with an SLE therapy compared to the SLE activity before treatment with an SLE therapy can indicate that the SLE therapy is effective. Assessing a mammal for SLE disease activity during treatment of the mammal for SLE can also allow responders to the SLE therapy to be identified. For example, a decrease in SLE activity in a mammal during treatment with an SLE therapy compared to the SLE activity in the mammal before treatment with the SLE therapy can indicate that the mammal is a responder to the SLE therapy.

This document also provides methods and materials for identifying mammals (e.g., humans) having SLE that are likely to experience SLE disease activity. For example, future SLE disease activity in a mammal can be predicted by determining whether or not the mammal's serum contains one or more of the polypeptides listed in Table 2, 3, or 4 at a level that is greater than or less than the average level of the same one or more polypeptides observed in control serum obtained from control mammals.

This document also provides methods and materials for identifying mammals (e.g., humans) likely to respond to an anti-IFN SLE treatment. For example, the methods and materials provided herein can be used to identify SLE patients having serum containing one or more IFN-regulated chemokine polypeptides at a level that is greater than or less than the average level of the same one or more polypeptides observed in control serum obtained from control mammals. Once identified, patients can be treated with an anti-IFN treatment such as humanized anti-IFN antibodies. In some cases, the effectiveness of the anti-IFN SLE treatment can be assessed as described herein.

This document also provides arrays for detecting polypeptides. The arrays provided herein can be two-dimensional arrays, and can contain at least two different polypeptides capable of detecting polypeptides, such as antibodies (e.g., at least three, at least five, at least ten, at least 20, at least 30, at least 50, at least 100, or at least 200 different polypeptides capable of detecting polypeptides). The arrays provided herein also can contain multiple copies of each of many different polypeptides. In addition, the arrays for detecting polypeptides provided herein can contain polypeptides attached to any suitable surface (e.g., plastic or glass).

A polypeptide capable of detecting a polypeptide can be naturally occurring, recombinant, or synthetic. The polypeptides immobilized on an array also can be antibodies or antibody fragments, such as Fab′ fragments, Fab fragments, single-chain Fvs, antigen-specific polyclonal antibodies, or full-length monoclonal antibodies. Such an antibody or antibody fragment can be capable of binding specifically to a polypeptide listed in Table 2, 3, or 4. The polypeptides immobilized on the array can be members of a family such as a receptor family, ligand family, or enzyme family.

The production of monoclonal antibodies against a polypeptide target is routine using standard hybridoma technology. In addition, numerous monoclonal antibodies are available commercially. An antibody fragment can be produced by any means. For example, an antibody fragment can be enzymatically or chemically produced by fragmentation of an intact antibody. An antibody fragment also can be produced synthetically or recombinantly from a gene encoding the partial antibody sequence. The antibody fragment can be a single chain antibody fragment. Alternatively, the fragment can include multiple chains which are linked together, for instance, by disulfide linkages. The fragment may also optionally be a multimolecular complex.

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

The invention will be further described in the following examples, which do not limit the scope of the invention described in the claims.

### EXAMPLES

### Example 1

### Identifying Polypeptides That Can Be Used as Biomarkers for SLE

SLE patients were enrolled from the Hopkins Lupus Cohort (Petri, Rheum Dis Clin North Am 26:199-213 (2000)). Healthy age- and gender-matched controls were recruited. Following informed consent, blood samples were collected with the PAXgene system (Qiagen/Becton-Dickinson, Hombrechtikon, Switzerland and Franklin Lakes, N.J.). These samples were used to generate whole blood gene expression profiles. cRNA probes were prepared from RNA purified using the PAXgene system. The cRNA probes were hybridized with Affymetrix U133A arrays (Affymetrix, Santa Clara, Calif.) using standard protocols. The levels of 82 IFN-regulated genes that distinguished 81 SLE patients from 42 healthy controls were normalized and used to assign a gene expression “score,” the IFN gene score, as described previously (Baechler et al., Proc Nat Acad Sci USA 100:2610-2615 (2003)).

Fifteen patients with high levels of IFN-regulated transcripts (IFN-hi), 15 patients with lower levels of the same transcripts (IFN-lo), and 15 matched controls were studied further. Clinical and demographic features of the two groups are compared in Table 1. The IFN-hi group was enriched for African-American women (8/15) compared with the IFN-lo group (2/15; P=0.05), and, on average, fulfilled more criteria for SLE (7.3) than IFN-lo cases (5.7; P=0.004). All of the IFN-hi cases had a history of positive anti-dsDNA Abs and either low C3 or C4 complement levels, while these features were found in only about half of the IFN-lo group. The IFN-hi group also showed evidence of more active disease than the IFN-lo group as determined by higher SLEDAI and SLAM-R scores, together with several laboratory measures characteristic of active disease (low complement C3, and elevated ESR and anti-dsDNA antibodies) at the time of the visit. There were no significant differences in medication profiles between the groups. Thus, disease severity appeared to be increased in IFN-hi as compared to IFN-lo patients.

TABLE 1
Clinical and demographic features of SLE cases

| | IFN-hi | IFN-lo | |
|---|---|---|---|
| | (N = 15) | (N = 15) | P |
| Age (avg.) | 37.7^{a} | 40.9 | |
| Female | 15^{b} | 13 | |
| Caucasian | 6 | 13 | 0.01 |
| African-American | 8 | 2 | 0.05 |
| Historical | | | |
| ACR criteria (avg. #) | 7.3 | 5.7 | 0.004 |
| Malar Rash | 11 | 7 | |
| Discoid Rash | 3 | 1 | |
| Photosensitivity | 8 | 6 | |
| Oral/Nasal Ulcers | 8 | 9 | |
| Arthritis | 12 | 12 | |
| Pleurisy/Pericarditis | 13 | 7 | 0.03 |
| Renal | 9 | 8 | |
| Neurologic | 3 | 0 | |
| Hematologic | 14 | 12 | |
| Immunologic | 15 | 8 | 0.003 |
| Positive ANA | 15 | 15 | |
| Hx low C3 | 15 | 7 | 0.001 |
| Hx low C4 | 14 | 6 | 0.003 |
| Hx anti-dsDNA Abs | 15 | 8 | 0.003 |
| Hx anti-Ro Abs | 8 | 3 | |
| Hx anti-La Abs | 4 | 0 | 0.05 |
| Current Visit | | | |
| PGA (0-3 scale) | 1.5 | 1.1 | |
| SLEDAI | 5.6 | 2.3 | 0.008 |
| BILAG | 4.8 | 3.9 | |
| SLAM-R | 5.9 | 2.8 | 0.0001 |
| HCT | 37.3 | 36.9 | |
| WBC count | 5.3 | 7.4 | |
| ESR | 51.3 | 19.7 | 0.008 |
| Complement C3 | 80.5 | 109.6 | 0.008 |
| Complement C4 | 13.9 | 18.6 | |
| Positive anti-DNA Abs | 8 | 1 | 0.007 |
| Platelet count | 264.4 | 181.7 | |
| Lymphocyte count | 0.8 | 1.6 | 0.013 |
| Prednisone (avg. dose | 13 (14.6) | 9 (21.9) | |
| mgs) | | | |
| IV steroids | 6 | 4 | |
| Immunosuppressives | 4 | 7 | |
| Steroids and/or | 14 | 11 | |
| immunosuppressives | | | |
| Plaquenil | 12 | 12 | |
| NSAID | 6 | 4 | |

^{a}Continuous variables were compared using unpaired T tests and results reported with one decimal place.
^{b}Data indicate the number of individuals positive for the feature within each group of 15. These variables were compared using Fisher's Exact Test.

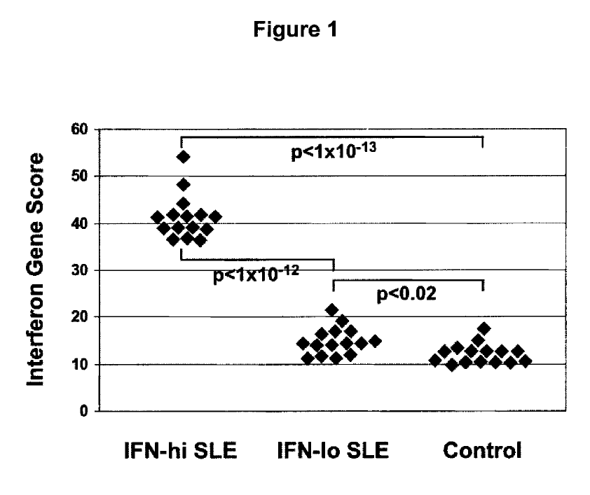

The IFN gene expression scores of the 45 study subjects are plotted in FIG. 1. Detailed clinical data were available for each visit, including the SLEDAI and SLAM disease activity measures, laboratory test results, and medication profiles. The SLEDAI (Bombardier et al., Arthritis Rheum 35:630-640 (1992)) consists of 22 defined weighted items grouped into nine organ systems. The index was calculated by summing all weighted items that were present within the previous ten days. Possible SLEDAI scores ranged from 0 to 101. The Systemic Lupus Activity Measure-Revised (SLAM-R) listed 33 clinical and laboratory manifestations of SLE (Liang et al., Arthritis Rheum 32:1107-1118 (1989)). Each manifestation was graded according to the severity of activity within the month before evaluation. Possible total scores vary from 0 to 86.

Serum was isolated from patients and control subjects by peripheral venipuncture using serum-separator vacutainer tubes (Becton-Dickinson, Franklin Lakes, N.J.). A protease inhibitor (aprotinin, 1 μg/mL) was added to each sample, and aliquots were immediately frozen at −80° C. The levels of 160 serum polypeptide analytes were measured in serum aliquots (100 μL) using custom dual-antibody sandwich immunoassay arrays, as described elsewhere (Shao et al., J Biomed Biotechnol 2003:299-307 (2003); Perlee et at., Proteome Sci 2:9 (2004)). Briefly, monoclonal capture antibodies specific for each analyte were fixed to glass slides, with 12 replicate spots for each analyte. The slides were incubated with serum samples for two hours. Each serum sample was tested in duplicate. Slides were washed and incubated with secondary biotinylated polyclonal antibodies. The signals were amplified using a “rolling circle” method (Perlee et al., Proteome Sci 2:9 (2004)).

Quality control measures included optimization of antibody pairs, use of internal controls to minimize array-to-array variation, and standardized procedures for array manufacturing (Shao et al., J Biomed Biotechnol 2003:299-307 (2003); Perlee et al., Proteome Sci 2:9 (2004)). Arrays were scanned using a Tecan LS200 scanner (Tecan, Männedorf, Switzerland), Mean Fluorescence Intensities (MFIs) were generated using customized software. MFIs were converted to concentration values using best-fit equations generated for each analyte using 15 serial dilutions of a known concentration of recombinant analyte. Four anchor-point control dilutions of recombinant analytes were included on each slide. The upper and lower limits of quantitation were defined to ensure a dynamic working range. The results are presented in Table 2. CXCL10 (IP-10) levels were measured using Luminex assays since an antibody directed against this analyte was not included on the antibody array. In addition to the 161 serum polypeptide analytes listed in Table 2, the following twelve analytes were measured using the antibody arrays: ANG, CNTF, D-DIMER DD5, D-DIMER DD6, IGFBP-3, IGFBP-6, IL-17, MMP9, PROTEIN C, PROTEIN S, TIMP-2, and VAP-1. These 12 analytes were excluded from further analysis because the concentrations present in ≧80% of the samples were at the upper or lower limits of detection.

TABLE 2
Mean Serum Analyte Concentrations (pg/ml) for 15 IFN-hi SLE Cases, 15 IFN-lo SLE Cases, and 15 Controls

| | | | | | | | | | All SLE v. | | IFN-hi v. | | IFN-lo v. | | IFN-hi v. | |
|---|---|---|---|---|---|---|---|---|---|---|---|---|---|---|---|---|
| | | Common | Accession | Gene | | | | | Ctrl | | Ctrl | | Ctrl | | IFN-lo | |
| | Analyte | name | No. | ID | All SLE Avg | IFN-hi Avg | IFN-lo Avg | Ctrl Avg | FC | p-val | FC | p-val | FC | p-val | FC | p-val |
| 1 | ACE | angiotensin I | NP_000780.1 | 1636 | 39757^{a} | 34268 | 45246 | 53205 | −1.34 | 9.1E−03 | −1.55 | 8.0E−04 | −1.18 | 1.9E−01 | −1.32 | 5.6E−02 |
| | | converting | | | | | | | | | | | | | | |
| | | enzyme 1 | | | | | | | | | | | | | | |
| 2 | ACE2 | angiotensin I | NP_068576.1 | 59272 | 2639 | 1968 | 3310 | 2871 | −1.09 | 5.6E−01 | −1.46 | 4.0E−03 | 1.15 | 4.8E−01 | −1.68 | 3.4E−02 |
| | | converting | | | | | | | | | | | | | | |
| | | enzyme 2 | | | | | | | | | | | | | | |
| 3 | BDNF | brain-derived | NP_001700.2 | 627 | 928 | 1182 | 673 | 764 | 1.21 | 2.1E−01 | 1.55 | 3.7E−02 | −1.14 | 4.0E−01 | 1.76 | 1.4E−02 |
| | | neurotrophic | | | | | | | | | | | | | | |
| | | factor | | | | | | | | | | | | | | |
| 4 | BLC | CXCL13 | NP_006410.1 | 10563 | 209 | 233 | 185 | 107 | 1.96 | 8.8E−04 | 2.18 | 3.4E−03 | 1.73 | 7.5E−02 | 1.26 | 3.7E−01 |
| 5 | EGF | epidermal | NP_001954.1 | 1950 | 61 | 62 | 59 | 29 | 2.12 | 1.8E−03 | 2.16 | 7.5E−03 | 2.08 | 1.0E−02 | 1.04 | 8.6E−01 |
| | | growth factor | | | | | | | | | | | | | | |
| 6 | FGFR3 | fibroblast | NP_000133.1 | 2261 | 632 | 625 | 639 | 934 | −1.48 | 9.3E−03 | −1.50 | 1.0E−02 | −1.46 | 1.7E−02 | −1.02 | 8.6E−01 |
| | (IIIC) | growth factor | | | | | | | | | | | | | | |
| | | receptor 3 | | | | | | | | | | | | | | |
| | | (IIIC) | | | | | | | | | | | | | | |
| 7 | FGF2 | fibroblast | NP_001997.4 | 2247 | 544 | 575 | 513 | 768 | −1.41 | 4.5E−04 | −1.34 | 2.2E−02 | −1.50 | 1.1E−04 | 1.12 | 4.1E−01 |
| | | growth factor 2 | | | | | | | | | | | | | | |
| 8 | GDNF | glial cell | NP_000505.1 | 2668 | 9 | 10 | 8 | 6 | 1.43 | 3.9E−04 | 1.53 | 9.2E−03 | 1.33 | 1.2E−02 | 1.15 | 3.3E−01 |
| | | derived | | | | | | | | | | | | | | |
| | | neurotrophic | | | | | | | | | | | | | | |
| | | factor | | | | | | | | | | | | | | |
| 9 | GROB | CXCL2 | NP_002080.1 | 2920 | 936 | 1091 | 780 | 513 | 1.82 | 1.7E−02 | 2.13 | 3.2E−02 | 1.52 | 2.5E−01 | 1.40 | 3.5E−01 |
| 10 | ICAM3 | intercellular | NP_002153.1 | 3385 | 9702 | 11519 | 7884 | 7394 | 1.31 | 8.4E−03 | 1.56 | 3.7E−03 | 1.07 | 5.6E−01 | 1.46 | 1.6E−02 |
| | | adhesion | | | | | | | | | | | | | | |
| | | molecule 3 | | | | | | | | | | | | | | |
| 11 | IFNW | interferon | NP_002168.1 | 3467 | 7548 | 7576 | 7519 | 14195 | −1.88 | 7.0E−04 | −1.87 | 9.7E−04 | −1.89 | 7.4E−04 | 1.01 | 9.6E−01 |
| | | omega | | | | | | | | | | | | | | |
| 12 | IL15 | interleukin 15 | NP_000576.1 | 3600 | 433 | 515 | 351 | 274 | 1.58 | 8.7E−03 | 1.88 | 4.3E−02 | 1.28 | 9.0E−04 | 1.47 | 1.6E−01 |
| 13 | IL18 | interleukin 18 | NP_001553.1 | 3606 | 129 | 151 | 108 | 88 | 1.46 | 4.2E−03 | 1.71 | 1.5E−03 | 1.22 | 2.5E−01 | 1.40 | 4.8E−02 |
| 14 | IL2SRA | interleukin 2 | NP_000408.1 | 3559 | 890 | 940 | 840 | 521 | 1.71 | 3.1E−05 | 1.81 | 1.1E−03 | 1.61 | 1.0E−03 | 1.12 | 4.1E−01 |
| | | receptor, | | | | | | | | | | | | | | |
| | | alpha | | | | | | | | | | | | | | |
| 15 | IL5 | interleukin 5 | NP_000870.1 | 3567 | 3 | 4 | 3 | 2 | 1.54 | 5.1E−04 | 1.72 | 7.4E−03 | 1.37 | 1.0E−02 | 1.26 | 1.8E−01 |
| 16 | IL6 | interleukin 6 | NP_000591.1 | 3569 | 10 | 11 | 9 | 3 | 2.83 | 9.1E−07 | 3.11 | 7.9E−05 | 2.55 | 3.2E−03 | 1.22 | 3.5E−01 |
| 17 | IL8 | interleukin 8 | NP_000575.1 | 3576 | 8 | 9 | 8 | 4 | 2.31 | 8.6E−05 | 2.49 | 4.5E−03 | 2.14 | 4.8E−03 | 1.16 | 5.3E−01 |
| 18 | IP10^{b} | CXCL10 | NP_001556.1 | 3627 | 77 | 120 | 23 | 13 | 5.82 | 1.4E−03 | 9.09 | 1.5E−03 | 1.74 | 2.6E−02 | 5.82 | 3.1E−03 |
| 19 | ITAC | CXCL11 | NP_005400.1 | 6373 | 204 | 263 | 145 | 167 | 1.23 | 1.5E−01 | 1.58 | 4.2E−02 | −1.15 | 2.1E−01 | 1.81 | 1.8E−02 |
| 20 | MCP1 | CCL2 | NP_002973.1 | 6347 | 92 | 135 | 50 | 24 | 3.78 | 8.5E−04 | 5.52 | 3.1E−03 | 2.04 | 5.7E−02 | 2.71 | 1.9E−02 |
| 21 | MCP2 | CCL8 | NP_005614.2 | 6355 | 38 | 51 | 26 | 15 | 2.48 | 9.1E−05 | 3.27 | 6.9E−04 | 1.69 | 2.2E−02 | 1.93 | 1.4E−02 |
| 22 | MCP3 | CCL7 | NP_006264.2 | 6354 | 48 | 50 | 46 | 31 | 1.57 | 9.6E−06 | 1.63 | 2.1E−04 | 1.51 | 1.5E−03 | 1.08 | 5.0E−01 |
| 23 | MIG | CXCL9 | NP_002407.1 | 4283 | 127 | 180 | 74 | 56 | 2.29 | 1.1E−02 | 3.24 | 1.4E−02 | 1.34 | 3.5E−01 | 2.42 | 3.9E−02 |
| 24 | MIP1A | CCL3 | | 6348 | 370 | 438 | 301 | 219 | 1.69 | 4.9E−07 | 2.00 | 1.8E−06 | 1.38 | 1.1E−03 | 1.45 | 6.5E−04 |
| 25 | MIP3A | CCL20 | NP_004582.1 | 6364 | 79 | 81 | 76 | 117 | −1.48 | 6.0E−03 | −1.43 | 1.1E−02 | −1.53 | 4.1E−03 | 1.06 | 4.0E−01 |
| 26 | MIP3B | CCL19 | NP_006265.1 | 6363 | 158 | 218 | 98 | 77 | 205 | 1.6E−04 | 2.82 | 9.0E−05 | 1.27 | 1.4E−01 | 2.22 | 5.1E−04 |
| 27 | MMP7 | matrix | NP_002414.1 | 4316 | 1252 | 1295 | 1209 | 790 | 1.58 | 2.1E−03 | 1.64 | 6.4E−03 | 1.53 | 8.0E−02 | 1.07 | 7.5E−01 |
| | | metalloproteinase 7 | | | | | | | | | | | | | | |
| 28 | PDGFRA | platelet- | NP_006197.1 | 5156 | 23663 | 24381 | 22945 | 35538 | −1.50 | 7.2E−04 | −1.46 | 2.4E−03 | −1.55 | 5.4E−04 | 1.06 | 5.6E−01 |
| | | derived | | | | | | | | | | | | | | |
| | | growth factor | | | | | | | | | | | | | | |
| | | receptor, | | | | | | | | | | | | | | |
| | | alpha | | | | | | | | | | | | | | |
| 29 | TARC | CCL17 | NP_002978.1 | 6361 | 68 | 74 | 63 | 42 | 1.61 | 2.3E−02 | 1.74 | 3.1E−02 | 1.48 | 2.4E−01 | 1.17 | 6.0E−01 |
| 30 | TGFBRIII | transforming | NP_003234.2 | 7049 | 16431 | 17611 | 15250 | 7541 | 2.18 | 1.8E−03 | 2.34 | 5.2E−03 | 2.02 | 2.6E−02 | 1.15 | 5.4E−01 |
| | | growth factor, | | | | | | | | | | | | | | |
| | | beta receptor | | | | | | | | | | | | | | |
| | | III | | | | | | | | | | | | | | |
| 31 | 41BB | TNFR | NP_001552.2 | 3604 | 208 | 195 | 221 | 235 | −1.13 | 3.7E−01 | −1.20 | 1.8E−0.1 | −1.06 | 7.3E−01 | −1.13 | 4.8E−01 |
| | | Superfamily, | | | | | | | | | | | | | | |
| | | member 9 | | | | | | | | | | | | | | |
| 32 | 6CKINE | CCL21 | NP_002980.1 | 6366 | 845 | 907 | 782 | 642 | 1.32 | 8.3E−05 | 1.41 | 2.8E−04 | 1.22 | 5.4E−03 | 1.16 | 5.8E−02 |
| 33 | AFP | alpha- | NP_001125.1 | 174 | 769 | 1089 | 448 | 496 | 1.55 | 4.1E−01 | 2.19 | 3.7E−01 | −1.11 | 5.4E−01 | 2.43 | 3.4E−01 |
| | | fetoprotein | | | | | | | | | | | | | | |
| 34 | AGRP | agouti related | NP_001129.1 | 181 | 213 | 216 | 211 | 254 | −1.19 | 3.0E−02 | −1.17 | 7.0E−02 | −1.20 | 4.2E−02 | 1.03 | 7.7E−01 |
| | | protein | | | | | | | | | | | | | | |
| | | homolog | | | | | | | | | | | | | | |
| | | (mouse) | | | | | | | | | | | | | | |
| 35 | ALCAM | activated | NP_001618.1 | 214 | 13364 | 14278 | 12450 | 15481 | −1.16 | 7.6E−03 | −1.08 | 2.2E−01 | −1.24 | 1.2E−03 | 1.15 | 8.6E−02 |
| | | leukocyte cell | | | | | | | | | | | | | | |
| | | adhesion | | | | | | | | | | | | | | |
| | | molecule | | | | | | | | | | | | | | |
| 36 | AR | amphiregulin | NP_001648.1 | 374 | 35 | 37 | 32 | 36 | −1.05 | 5.2E−01 | 1.01 | 9.2E−01 | −1.12 | 1.5E−01 | 1.13 | 1.5E−01 |
| 37 | BNGF | nerve growth | NP_002497.1 | 4803 | 1109 | 1156 | 1062 | 1234 | −1.11 | 1.6E−01 | −1.07 | 5.0E−01 | −1.16 | 6.8E−02 | 1.09 | 4.0E−01 |
| | | factor, beta | | | | | | | | | | | | | | |
| | | protein | | | | | | | | | | | | | | |
| 38 | BTC | betacellulin | NP_001720.1 | 685 | 703 | 772 | 633 | 761 | −1.08 | 5.3E−01 | 1.01 | 9.4E−01 | −1.20 | 1.3E−01 | 1.22 | 3.8E−01 |
| 39 | CA125 | mucin 16 | | 94025 | 530 | 505 | 555 | 718 | −1.35 | 7.9E−02 | −1.42 | 5.2E−02 | −1.29 | 1.7E−01 | −1.10 | 5.3E−01 |
| 40 | CD141 | thrombomodulin | NP_000352.1 | 7056 | 15308 | 14839 | 15776 | 18667 | −1.22 | 6.2E−02 | −1.26 | 9.1E−02 | −1.18 | 1.7E−01 | −1.06 | 6.9E−01 |
| 41 | CD27 | TNFR | NP_001233.1 | 939 | 5116 | 5752 | 4480 | 4958 | 1.03 | 6.5E−01 | 1.16 | 1.7E−01 | −1.11 | 1.3E−01 | 1.28 | 4.4E−02 |
| | | superfamily, | | | | | | | | | | | | | | |
| | | member 7 | | | | | | | | | | | | | | |
| 42 | CD30 | TNFR | NP_001234.1 | 943 | 4991 | 4971 | 5010 | 5873 | −1.18 | 2.7E−02 | −1.18 | 1.1E−01 | −1.17 | 5.1E−02 | −1.01 | 9.5E−01 |
| | | superfamily, | | | | | | | | | | | | | | |
| | | member 8 | | | | | | | | | | | | | | |
| 43 | CD40 | TNFR | NP_001241.1 | 958 | 330 | 328 | 332 | 345 | −1.05 | 6.3E−01 | −1.05 | 6.3E−01 | −1.04 | 7.2E−01 | −1.01 | 9.0E−01 |
| | | superfamily | | | | | | | | | | | | | | |
| | | member 5 | | | | | | | | | | | | | | |
| 44 | CD44V6 | CD44 antigen | NP_000601.3 | 960 | 14912 | 14651 | 15173 | 17100 | −1.15 | 2.5E−01 | −1.17 | 1.9E−01 | −1.13 | 3.3E−01 | −1.04 | 4.8E−01 |
| 45 | CNTF RA | ciliary | | 12803 | 253 | 244 | 262 | 302 | −1.19 | 1.9E−03 | −1.24 | 9.2E−04 | −1.15 | 7.8E−02 | −1.07 | 4.4E−01 |
| | | neurotrophic | | | | | | | | | | | | | | |
| | | factor | | | | | | | | | | | | | | |
| 46 | CRP | c-reactive | NP_000558.2 | 1401 | 10184 | 10229 | 10139 | 10850 | −1.07 | 2.3E−01 | −1.06 | 3.7E−01 | −1.07 | 2.8E−01 | 1.01 | 9.0E−01 |
| | | protein | | | | | | | | | | | | | | |
| 47 | CTACK | CCL27 | NP_006655.1 | 10850 | 718 | 692 | 744 | 901 | −1.25 | 7.7E−03 | −1.30 | 4.0E−03 | −1.21 | 7.1E−02 | −1.07 | 5.2E−01 |
| 48 | DR6 | TNFR | NP_055267.1 | 27242 | 2831 | 2977 | 2684 | 2508 | 1.13 | 1.8E−01 | 1.19 | 2.1E−01 | 1.07 | 4.7E−01 | 1.11 | 4.6E−01 |
| | | superfamily, | | | | | | | | | | | | | | |
| | | member 21 | | | | | | | | | | | | | | |
| 49 | ENA78 | CXCL5 | NP_002985.1 | 6374 | 3035 | 3974 | 2096 | 2470 | 1.23 | 4.2E−01 | 1.61 | 1.6E−01 | −1.18 | 6.3E−01 | 1.90 | 1.3E−01 |
| 50 | ENDOSTATIN | procollagen, | NP_085059.1 | 80781 | 8586 | 8193 | 8978 | 9916 | −1.15 | 7.1E−02 | −1.21 | 5.2E−02 | −1.10 | 2.9E−01 | −1.10 | 4.2E−01 |
| | | type XVIII, | | | | | | | | | | | | | | |
| | | alpha 1 | | | | | | | | | | | | | | |
| 51 | EOT | CCL11 | NP_002977.1 | 6356 | 142 | 142 | 142 | 114 | 1.25 | 1.1E−01 | 1.25 | 1.7E−01 | 1.25 | 2.3E−01 | −1.00 | 1.0E+00 |
| 52 | EOT2 | CCL24 | NP_002982.2 | 6369 | 335 | 229 | 441 | 488 | −1.46 | 3.2E−01 | −2.31 | 9.2E−02 | −1.11 | 7.9E−01 | −1.92 | 6.3E−02 |
| 53 | EOT3 | CCL26 | NP_006063.1 | 10344 | 590 | 612 | 568 | 745 | −1.26 | 7.0E−04 | −1.22 | 2.1E−02 | −1.31 | 1.1E−03 | 1.08 | 4.6E−01 |
| 54 | ERBB1 | epidermal | NP_005219.2 | 1956 | 15201 | 13979 | 16423 | 18595 | −1.22 | 9.1E−03 | −1.33 | 4.4E−03 | −1.13 | 1.5E−01 | −1.17 | 1.5E−01 |
| | | growth factor | | | | | | | | | | | | | | |
| | | receptor | | | | | | | | | | | | | | |
| 55 | ERBB2 | v-erb-b2 | NP_001005862.1 | 2064 | 3910 | 3647 | 4173 | 4905 | −1.25 | 1.1E−03 | −1.34 | 2.5E−04 | −1.18 | 2.3E−02 | −1.14 | 7.0E−02 |
| | | erythroblastic | | | | | | | | | | | | | | |
| | | leukemia | | | | | | | | | | | | | | |
| | | homolog 2 | | | | | | | | | | | | | | |
| 56 | ESELECTIN | selectin E | NP_000441.1 | 6401 | 5889 | 6567 | 5210 | 6034 | −1.02 | 8.5E−01 | 1.09 | 5.9E−01 | −1.16 | 2.9E−01 | 1.26 | 1.1E−01 |
| 57 | ET3 | endothelin | NP_000105.1 | 1908 | 2414 | 2455 | 2373 | 2867 | −1.19 | 1.9E−01 | −1.17 | 2.5E−01 | −1.21 | 1.8E−01 | 1.03 | 7.6E−01 |
| 58 | FAS | TNFR | NP_000034.1 | 355 | 558 | 596 | 520 | 1734 | −3.11 | 3.7E−01 | −2.91 | 3.8E−01 | −3.34 | 3.5E−01 | 1.15 | 2.7E−01 |
| | | superfamily, | | | | | | | | | | | | | | |
| | | member 6 | | | | | | | | | | | | | | |
| 59 | FASL | fas ligand | NP_000630.1 | 356 | 1975 | 2032 | 1918 | 2542 | −1.29 | 8.3E−02 | −1.25 | 1.6E−01 | −1.33 | 6.6E−02 | 1.06 | 6.6E−01 |
| 60 | FGF1 | fibroblast | NP_000791.1 | 2246 | 91 | 90 | 91 | 89 | 1.02 | 8.6E−01 | 1.01 | 9.3E−01 | 1.02 | 8.6E−01 | −1.01 | 9.2E−01 |
| | | growth factor 1 | | | | | | | | | | | | | | |
| 61 | FGF4 | fibroblast | NP_001998.1 | 2249 | 815 | 824 | 806 | 1117 | −1.37 | 1.3E−03 | −1.36 | 4.6E−03 | −1.39 | 1.6E−03 | 1.02 | 8.2E−01 |
| | | growth factor 4 | | | | | | | | | | | | | | |
| 62 | FGF6 | fibroblast | NP_066276.2 | 2251 | 518 | 503 | 532 | 619 | −1.20 | 7.7E−02 | −1.23 | 9.3E−02 | −1.16 | 1.6E−01 | −1.06 | 6.6E−01 |
| | | growth factor 6 | | | | | | | | | | | | | | |
| 63 | FGF7 | fibroblast | NP_002000.1 | 2252 | 221 | 232 | 211 | 231 | −1.40 | 7.1E−01 | 1.00 | 9.8E−01 | −1.09 | 4.6E−01 | 1.10 | 3.4E−01 |
| | | growth factor 7 | | | | | | | | | | | | | | |
| 64 | FGFR3(IIIB) | fibroblast | NP_000133.1 | 2261 | 865 | 860 | 871 | 1011 | −1.17 | 1.2E−01 | −1.18 | 1.4E−01 | −1.16 | 1.8E−01 | −1.01 | 9.1E−01 |
| | | growth factor | | | | | | | | | | | | | | |
| | | receptor 3 | | | | | | | | | | | | | | |
| | | (IIIB) | | | | | | | | | | | | | | |
| 65 | FLT3LIG | fms-related | NP_001450.2 | 2323 | 132 | 157 | 107 | 121 | 1.09 | 4.5E−01 | 1.30 | 1.1E−01 | −1.13 | 2.7E−01 | 1.47 | 3.7E−02 |
| | | tyrosine | | | | | | | | | | | | | | |
| | | kinase 3 | | | | | | | | | | | | | | |
| | | ligand | | | | | | | | | | | | | | |
| 66 | FOLLISTATIN | follistatin | NP_006341.1 | 10468 | 1958 | 1547 | 2369 | 1696 | 1.15 | 5.4E−01 | −1.10 | 2.6E−01 | 1.40 | 4.2E−01 | −1.53 | 3.3E−01 |
| 67 | FRACTALKINE | CX3CL1 | NP_002987.1 | 6376 | 752 | 688 | 816 | 852 | −1.13 | 4.0E−01 | −1.24 | 1.4E−01 | −1.04 | 8.4E−01 | −1.19 | 4.4E−01 |
| 68 | GCP2 | CXCL6 | NP_002984.1 | 6372 | 253 | 325 | 180 | 207 | 1.22 | 3.8E−01 | 1.57 | 2.1E−01 | −1.15 | 5.2E−01 | 1.81 | 1.5E−01 |
| 69 | GCSF | colony | NP_000750.1 | 1440 | 1220 | 1235 | 1205 | 1252 | −1.03 | 7.9E−01 | −1.01 | 9.1E−01 | −1.04 | 7.3E−01 | 1.02 | 8.4E−01 |
| | | stimulating | | | | | | | | | | | | | | |
| | | factor 3 | | | | | | | | | | | | | | |
| 70 | GMCSF | colony | NP_000749.2 | 1437 | 27 | 30 | 24 | 23 | 1.19 | 1.1E−01 | 1.32 | 4.0E−02 | 1.05 | 6.5E−01 | 1.26 | 7.2E−02 |
| | | stimulating | | | | | | | | | | | | | | |
| | | factor 2 | | | | | | | | | | | | | | |
| 71 | GROG | CXCL3 | NP_002081.2 | 2921 | 901 | 1195 | 607 | 741 | 1.22 | 3.3E−01 | 1.61 | 1.5E−01 | −1.22 | 5.5E−02 | 1.97 | 6.7E−02 |
| 72 | HBEGF | heparin- | NP_001936.1 | 1839 | 94 | 90 | 98 | 104 | −1.11 | 2.4E−01 | −1.16 | 1.8E−01 | −1.06 | 5.6E−01 | −1.10 | 4.6E−01 |
| | | binding EGF- | | | | | | | | | | | | | | |
| | | like growth | | | | | | | | | | | | | | |
| | | factor | | | | | | | | | | | | | | |
| 73 | HCC1 | RNA-binding | NP_004893.1 | 9584 | 5713 | 5560 | 5865 | 5514 | 1.04 | 3.6E−01 | 1.01 | 8.5E−01 | 1.06 | 2.3E−01 | −1.05 | 3.4E−01 |
| | | region | | | | | | | | | | | | | | |
| | | containing 2 | | | | | | | | | | | | | | |
| 74 | HCC4 | CCL16 | NP_004581.1 | 6360 | 4651 | 4921 | 4381 | 4758 | −1.02 | 8.2E−01 | 1.03 | 7.7E−01 | −1.09 | 4.7E−01 | 1.12 | 3.6E−01 |
| 75 | HCG | human | NP_000726.1 | 1081 | 1491 | 2364 | 619 | 745 | 2.00 | 4.0E−01 | 3.17 | 3.7E−01 | −1.20 | 2.9E−02 | 3.82 | 3.3E−01 |
| | | chorionic | | | | | | | | | | | | | | |
| | | gonadotropin | | | | | | | | | | | | | | |
| 76 | HGF | hepatocyte | NP_000592.3 | 3082 | 919 | 841 | 997 | 923 | −1.00 | 9.8E−01 | −1.10 | 5.9E−01 | 1.08 | 6.5E−01 | −1.19 | 3.9E−01 |
| | | growth factor | | | | | | | | | | | | | | |
| 77 | HSP70 | heat shock | NP_005336.2 | 3303 | 16077 | 16000 | 16154 | 19234 | −1.20 | 1.4E−01 | −1.20 | 1.6E−01 | −1.19 | 1.8E−01 | −1.01 | 9.3E−01 |
| | | protein 70 | | | | | | | | | | | | | | |
| 78 | HVEM | TNFR | NP_003811.2 | 8764 | 1938 | 1938 | 1938 | 1696 | 1.14 | 2.0E−01 | 1.14 | 3.8E−01 | 1.14 | 2.8E−01 | 1.00 | 1.0E+00 |
| | | superfamily, | | | | | | | | | | | | | | |
| | | member 14 | | | | | | | | | | | | | | |
| 79 | I309 | CCL1 | NP_002972.1 | 6346 | 26 | 29 | 24 | 29 | −1.10 | 3.7E−01 | −100 | 9.8E−01 | −1.23 | 9.8E−02 | 1.22 | 4.7E−02 |
| 80 | ICAM1 | intercellular | NP_000192.1 | 3383 | 8461 | 8729 | 8193 | 9319 | −1.10 | 6.0E−01 | −1.07 | 7.5E−01 | −1.14 | 5.1E−01 | 1.07 | 7.2E−01 |
| | | adhesion | | | | | | | | | | | | | | |
| | | molecule 1 | | | | | | | | | | | | | | |
| 81 | lFNA | interferon | NP_076918.1 | 3439 | 29 | 24 | 34 | 27 | 1.10 | 6.8E−01 | −1.10 | 6.4E−01 | 1.29 | 4.6E−01 | 1.41 | 3.3E−01 |
| | | alpha | | | | | | | | | | | | | | |
| 82 | IFNG | interferon, | | 3458 | 62 | 62 | 61 | 74 | −1.21 | 7.6E−02 | −1.21 | 1.3E−01 | −1.21 | 1.2E−01 | 1.00 | 9.8E−01 |
| | | gamma | | | | | | | | | | | | | | |
| 83 | IGFBP1 | insulin-like | NP_000587.1 | 3484 | 33784 | 39396 | 28171 | 34702 | −1.03 | 9.3E−01 | 1.14 | 7.0E−01 | −1.23 | 5.7E−01 | 1.40 | 3.2E−01 |
| | | growth factor | | | | | | | | | | | | | | |
| | | binding | | | | | | | | | | | | | | |
| | | protein 1 | | | | | | | | | | | | | | |
| 84 | IGFBP2 | insulin-like | NP_000588.2 | 3485 | 50266 | 51042 | 49490 | 48658 | 1.03 | 5.7E−01 | 1.05 | 4.3E−01 | 1.02 | 8.1E−01 | 1.03 | 6.3E−01 |
| | | growth factor | | | | | | | | | | | | | | |
| | | binding | | | | | | | | | | | | | | |
| | | protein 2 | | | | | | | | | | | | | | |
| 85 | IGFBF4 | insulin-like | NP_001543.1 | 3487 | 13884 | 13535 | 14233 | 15258 | −1.10 | 1.7E−01 | −1.13 | 1.4E−01 | −1.07 | 4.0E−01 | −1.05 | 5.9E−01 |
| | | growth factor | | | | | | | | | | | | | | |
| | | binding | | | | | | | | | | | | | | |
| | | protein 4 | | | | | | | | | | | | | | |
| 86 | IGFII | insulin-like | NP_000603.1 | 3481 | 2449 | 2704 | 2193 | 3050 | −1.25 | 7.2E−02 | −1.13 | 4.3E−01 | −1.39 | 1.1E−02 | 1.23 | 2.0E−01 |
| | | growth factor 2 | | | | | | | | | | | | | | |
| 87 | IGFIR | insulin-like | NP_000866.1 | 3480 | 947 | 903 | 991 | 1278 | −1.35 | 3.4E−02 | −1.41 | 3.6E−02 | −1.29 | 7.7E−02 | −1.10 | 5.4E−01 |
| | | growth factor | | | | | | | | | | | | | | |
| | | 1 receptor | | | | | | | | | | | | | | |
| 88 | IL10RB | interleukin 10 | NP_000619.3 | 3588 | 226 | 226 | 225 | 255 | −1.13 | 2.1E−02 | −1.13 | 1.5E−01 | −1.13 | 2.9E−02 | 1.00 | 9.7E−01 |
| | | receptor, beta | | | | | | | | | | | | | | |
| 89 | IL12P40 | interlukin | NP_002178.2 | 3593 | 1345 | 1389 | 1300 | 1577 | −1.17 | 1.0E−01 | −1.13 | 2.2E−01 | −1.21 | 1.1E−01 | 1.07 | 5.7E−01 |
| | | 12B | | | | | | | | | | | | | | |
| 90 | IL13 | interleukin 13 | NP_002179.2 | 3596 | 35 | 36 | 34 | 32 | 1.08 | 4.2E−01 | 1.10 | 4.0E−01 | 1.05 | 6.4E−01 | 1.05 | 6.5E−01 |
| 91 | IL16 | interleukin 16 | NP_004504.3 | 3603 | 1541 | 1538 | 1545 | 1670 | −1.08 | 1.9E−01 | −1.09 | 3.0E−01 | −1.08 | 3.2E−01 | −1.00 | 9.6E−01 |
| 92 | IL1A | interleukin 1, | NP_000566.3 | 3552 | 7 | 7 | 6 | 6 | 1.04 | 6.4E−01 | 1.10 | 2.5E−01 | −1.03 | 7.6E−01 | 1.13 | 9.7E−01 |
| | | alpha | | | | | | | | | | | | | | |
| 93 | IL1B | interleukin 1, | NP_000567.1 | 3553 | 6 | 7 | 6 | 6 | 1.09 | 3.1E−01 | 1.16 | 1.2E−01 | 1.01 | 9.1E−01 | 1.15 | 1.6E−01 |
| | | beta | | | | | | | | | | | | | | |
| 94 | IL1RA | interleukin 1 | NP_000568.1 | 3557 | 77 | 81 | 73 | 56 | 1.39 | 3.5E−03 | 1.47 | 1.9E−03 | 1.32 | 1.3E−01 | 1.11 | 5.2E−01 |
| | | receptor | | | | | | | | | | | | | | |
| | | antagonist | | | | | | | | | | | | | | |
| 95 | IL1SR1 | interleukin 1 | NP_000868.1 | 3554 | 4374 | 4598 | 4150 | 5526 | −1.26 | 1.1E−03 | −1.20 | 7.7E−02 | −1.33 | 1.4E−04 | 1.11 | 3.9E−01 |
| | | receptor type 1 | | | | | | | | | | | | | | |
| 96 | IL1SRII | interleukin 1 | NP_004624.1 | 7850 | 3692 | 3721 | 3662 | 5262 | −1.43 | 1.4E−04 | −1.41 | 5.2E−04 | −1.44 | 1.6E−04 | 1.02 | 8.5E−01 |
| | | receptor type 2 | | | | | | | | | | | | | | |
| 97 | IL2 | interleukin 2 | NP_000577.2 | 3558 | 3 | 4 | 3 | 3 | 1.05 | 8.1E−02 | 1.10 | 8.1E−02 | 1.00 | 3.3E−01 | 1.10 | 8.1E−02 |
| 98 | IL2RB | interleukin 2 | NP_000869.1 | 3560 | 9845 | 9852 | 9838 | 11478 | −1.17 | 4.6E−02 | −1.17 | 1.5E−01 | −1.17 | 7.8E−02 | 1.00 | 9.9E−01 |
| | | receptor, beta | | | | | | | | | | | | | | |
| 99 | IL2RG | interleukin 2 | NP_000197.1 | 3561 | 436 | 434 | 439 | 535 | −1.23 | 8.6E−02 | −1.23 | 1.1E−01 | −1.22 | 1.7E−01 | −1.01 | 9.5E−01 |
| | | receptor, | | | | | | | | | | | | | | |
| | | gamma | | | | | | | | | | | | | | |
| 100 | IL3 | interleukin 3 | NP_000579.2 | 3562 | 157 | 169 | 144 | 168 | −1.07 | 5.2E−01 | 1.01 | 9.5E−01 | −1.17 | 1.8E−01 | 1.17 | 1.8E−01 |
| 101 | IL4 | interleukin 4 | NP_000580.1 | 3565 | 34 | 34 | 34 | 35 | −1.04 | 6.3E−01 | −1.04 | 7.2E−01 | −1.04 | 6.2E−01 | 1.01 | 9.4E−01 |
| 102 | IL5RA | interleukin 5 | NP_000555.2 | 3568 | 1073 | 1037 | 1109 | 1318 | −1.23 | 8.0E−02 | −1.27 | 1.1E−01 | −1.19 | 1.9E−01 | −1.07 | 7.0E−01 |
| | | receptor, | | | | | | | | | | | | | | |
| | | alpha | | | | | | | | | | | | | | |
| 103 | IL7 | interleukin 7 | NP_000871.1 | 3574 | 11 | 12 | 10 | 9 | 1.23 | 4.3E−02 | 1.36 | 2.4E−02 | 1.11 | 3.6E−01 | 1.22 | 1.2E−01 |
| 104 | IL9 | interleukin 9 | NP_000581.1 | 3578 | 5129 | 5059 | 5199 | 5611 | −1.09 | 4.5E−01 | −1.11 | 5.0E−01 | −1.08 | 5.9E−01 | −1.03 | 8.8E−01 |
| 105 | LEPTIN | leptin | NP_000221.1 | 3952 | 61316 | 61457 | 61176 | 52604 | 1.17 | 2.0E−01 | 1.17 | 2.4E−01 | 1.16 | 2.4E−01 | 1.00 | 9.6E−01 |
| 106 | LIF | leukemia | NP_002300.1 | 3976 | 682 | 690 | 675 | 745 | −1.09 | 3.3E−01 | −1.08 | 4.8E−01 | −1.10 | 3.0E−01 | 1.02 | 8.2E−01 |
| | | inhibitory | | | | | | | | | | | | | | |
| | | factor | | | | | | | | | | | | | | |
| 107 | LIFRA | leukemia | NP_002301.1 | 3977 | 6449 | 6887 | 6012 | 6707 | −1.04 | 6.6E−01 | 1.03 | 8.2E−01 | −1.12 | 3.4E−01 | 1.15 | 3.5E−01 |
| | | inhibitory | | | | | | | | | | | | | | |
| | | factor | | | | | | | | | | | | | | |
| | | receptor | | | | | | | | | | | | | | |
| 108 | LSELECTIN | selectin L | NP_000646.1 | 6402 | 24767 | 25141 | 24393 | 24365 | 1.02 | 7.1E−01 | 1.03 | 5.0E−01 | 1.00 | 9.9E−01 | 1.03 | 7.0E−01 |
| 109 | LTBR | TNFR | NP_002333.1 | 4055 | 518 | 543 | 493 | 513 | 1.01 | 9.2E−01 | 1.06 | 7.1E−01 | −1.04 | 6.5E−01 | 1.10 | 5.5E−01 |
| | | superfamily, | | | | | | | | | | | | | | |
| | | member 3 | | | | | | | | | | | | | | |
| 110 | LYMPHOTACTIN | XCL1 | NP_002986.1 | 6375 | 632 | 653 | 611 | 814 | −1.29 | 5.4E−03 | −1.25 | 2.9E−02 | −1.33 | 3.3E−03 | 1.07 | 4.8E−01 |
| 111 | MCP4 | CCL13 | NP_005399.1 | 6357 | 219 | 223 | 215 | 222 | −1.01 | 8.8E−01 | 1.00 | 9.7E−01 | −1.03 | 7.7E−01 | 1.04 | 7.8E−01 |
| 112 | MCSF | colony | NP_000748.3 | 1435 | 138 | 155 | 122 | 149 | −1.08 | 4.4E−01 | 1.04 | 7.6E−01 | −1.23 | 5.0E−02 | 1.27 | 2.7E−02 |
| | | stimulating | | | | | | | | | | | | | | |
| | | factor 1 | | | | | | | | | | | | | | |
| 113 | MCSFR | colony | NP_005202.2 | 1436 | 53897 | 55653 | 52141 | 55491 | −1.03 | 6.8E−01 | 1.00 | 9.7E−01 | −1.06 | 5.1E−01 | 1.07 | 5.1E−01 |
| | | stimulating | | | | | | | | | | | | | | |
| | | factor 1 | | | | | | | | | | | | | | |
| | | receptor | | | | | | | | | | | | | | |
| 114 | MIF | macrophage | NP_002406.1 | 4282 | 50594 | 46404 | 54784 | 46258 | 1.09 | 1.7E−01 | 1.00 | 9.7E−01 | 1.18 | 3.4E−02 | −1.18 | 6.9E−02 |
| | | migration | | | | | | | | | | | | | | |
| | | inhibitory | | | | | | | | | | | | | | |
| | | factor | | | | | | | | | | | | | | |
| 115 | MIP1B | CCL4 | NP_002975.1 | 6351 | 40 | 69 | 11 | 10 | 4.06 | 1.8E−01 | 7.02 | 1.9E−01 | 1.10 | 6.5E−01 | 6.36 | 1.9E−01 |
| 116 | MIP1D | CCL15 | | 6359 | 922 | 943 | 901 | 1006 | −1.09 | 6.6E−01 | −1.07 | 7.8E−01 | −1.12 | 6.5E−01 | 1.05 | 8.6E−01 |
| 117 | MMP1 | matrix | NP_002412.1 | 4312 | 1645 | 1780 | 1510 | 1744 | −1.06 | 6.5E−01 | 1.02 | 8.9E−01 | −1.15 | 4.1E−01 | 1.18 | 4.0E−01 |
| | | metalloproteinase 1 | | | | | | | | | | | | | | |
| 118 | MMP10 | matrix | NP_002416.1 | 4319 | 1828 | 1994 | 1662 | 1728 | 1.06 | 4.0E−01 | 1.15 | 1.5E−01 | −1.04 | 5.5E−01 | 1.20 | 9.3E−02 |
| | | metalloproteinase | | | | | | | | | | | | | | |
| | | 10 | | | | | | | | | | | | | | |
| 119 | MMP2 | matrix | NP_004521.1 | 4313 | 24751 | 24283 | 25187 | 25324 | −1.02 | 8.0E−01 | −1.04 | 6.9E−01 | −1.01 | 9.7E−01 | −1.04 | 8.2E−01 |
| | | metalloproteinase 2 | | | | | | | | | | | | | | |
| 120 | MMP8 | matrix | NP_002415.1 | 4317 | 17360 | 14133 | 20587 | 19345 | −1.11 | 4.5E−01 | −1.37 | 9.6E−02 | 1.06 | 7.0E−01 | −1.46 | 7.8E−02 |
| | | metalloproteinase 8 | | | | | | | | | | | | | | |
| 121 | MPIF1 | CCL23 | NP_005055.2 | 6368 | 511 | 484 | 538 | 412 | 1.24 | 6.2E−02 | 1.17 | 3.7E−01 | 1.31 | 2.7E−02 | −1.11 | 5.4E−01 |
| 122 | NAP2 | CXCL7 | NP_002695.1 | 5473 | 9917 | 9888 | 9947 | 10197 | −1.03 | 3.9E−01 | −1.03 | 4.6E−01 | −1.03 | 5.1E−01 | −1.01 | 9.0E−01 |
| 123 | NEUTELAST | elastase 2, | NP_001963.1 | 1991 | 16767 | 15699 | 17836 | 15248 | 1.10 | 2.6E−01 | 1.03 | 7.8E−01 | 1.17 | 1.6E−01 | −1.14 | 3.1E−01 |
| | | neutrophil | | | | | | | | | | | | | | |
| 124 | NT3 | neurotrophin 3 | NP_002518.1 | 4908 | 128 | 135 | 121 | 151 | −1.18 | 9.7E−02 | −1.12 | 2.8E−01 | −1.25 | 3.0E−02 | 1.12 | 5.8E−02 |
| 125 | NT4 | neurotrophin 4 | NP_006170.1 | 4909 | 124 | 141 | 108 | 147 | −1.19 | 1.9E−01 | −1.05 | 7.4E−01 | −1.37 | 2.7E−02 | 1.31 | 1.9E−02 |
| 126 | OPN | osteopontin | NP_000573.1 | 6696 | 6685 | 6704 | 6666 | 5352 | 1.25 | 4.9E−02 | 1.25 | 1.4E−01 | 1.25 | 8.3E−02 | 1.01 | 9.7E−01 |
| 127 | OSM | oncostatin M | NP_065391.1 | 5008 | 21 | 24 | 18 | 18 | 1.16 | 1.9E−01 | 1.31 | 1.9E−01 | 1.00 | 3.3E−01 | 1.31 | 2.0E−01 |
| 128 | PAI1 | plasminogen | NP_000593.1 | 5054 | 8847 | 8961 | 8733 | 9410 | −1.06 | 4.5E−02 | −1.05 | 1.4E−01 | −1.08 | 1.6E−01 | 1.03 | 6.8E−01 |
| | | activator | | | | | | | | | | | | | | |
| | | inhibitor type 1 | | | | | | | | | | | | | | |
| 129 | PAIII | serine | NP_002566.1 | 5055 | 1128 | 1444 | 811 | 1026 | 1.10 | 7.5E−01 | 1.41 | 5.1E−01 | −1.26 | 1.2E−03 | 1.78 | 3.3E−01 |
| | | proteinase | | | | | | | | | | | | | | |
| | | inhibitor, | | | | | | | | | | | | | | |
| | | member 2 | | | | | | | | | | | | | | |
| 130 | PARC | p53- | NP_055904.1 | 23113 | 948 | 1001 | 896 | 1088 | −1.15 | 3.0E−01 | −1.09 | 5.5E−01 | −1.22 | 1.6E−01 | 1.12 | 2.6E−01 |
| | | associated | | | | | | | | | | | | | | |
| | | parkin-like | | | | | | | | | | | | | | |
| | | cytoplasmic | | | | | | | | | | | | | | |
| | | protein | | | | | | | | | | | | | | |
| 131 | PDGFRB | platelet- | NP_002600.1 | 5159 | 948 | 874 | 1023 | 1211 | −1.28 | 5.5E−02 | −1.39 | 2.4E−02 | −1.18 | 1.9E−01 | −1.17 | 1.8E−01 |
| | | derived | | | | | | | | | | | | | | |
| | | growth factor | | | | | | | | | | | | | | |
| | | receptor, beta | | | | | | | | | | | | | | |
| 132 | PECAM1 | platelet/endothelial | NP_000433.2 | 5175 | 7336 | 7764 | 6908 | 6065 | 1.21 | 1.7E−02 | 1.28 | 1.2E−02 | 1.14 | 1.9E−01 | 1.12 | 2.5E−01 |
| | | cell | | | | | | | | | | | | | | |
| | | adhesion | | | | | | | | | | | | | | |
| | | molecule | | | | | | | | | | | | | | |
| 133 | PEDF | pigment | NP_002606.3 | 5176 | 66553 | 63537 | 69570 | 67059 | −1.01 | 9.0E−01 | −1.06 | 4.1E−01 | 1.04 | 5.8E−01 | −1.09 | 1.5E−01 |
| | | epithelium | | | | | | | | | | | | | | |
| | | derived | | | | | | | | | | | | | | |
| | | factor, | | | | | | | | | | | | | | |
| | | member 1 | | | | | | | | | | | | | | |
| 134 | PF4 | CXCL4 | NP_002610.1 | 5196 | 3072 | 3007 | 3137 | 3412 | −1.11 | 5.2E−02 | −1.13 | 4.2E−02 | −1.09 | 2.2E−01 | −1.04 | 5.7E−01 |
| 135 | PLGF | placental | NP_002623.2 | 5228 | 37 | 40 | 34 | 28 | 1.32 | 2.6E−03 | 1.42 | 9.8E−04 | 1.22 | 4.6E−02 | 1.17 | 6.4E−02 |
| | | growth factor | | | | | | | | | | | | | | |
| 136 | PROLACTIN | prolactin | NP_000939.1 | 5617 | 13500 | 15277 | 11723 | 10551 | 1.28 | 1.1E−01 | 1.45 | 1.5E−01 | 1.11 | 4.8E−01 | 1.30 | 3.1E−01 |
| 137 | PSELECTIN | CD62P | NP_002996.1 | 6403 | 36020 | 33455 | 38586 | 44403 | −1.23 | 2.5E−01 | −1.33 | 1.9E−01 | −1.15 | 4.8E−01 | −1.15 | 5.0E−01 |
| 138 | RANK | TNFR | NP_003830.1 | 8792 | 245 | 217 | 273 | 252 | −1.03 | 8.1E−01 | −1.16 | 2.9E−01 | 1.08 | 6.2E−01 | −1.26 | 2.3E−01 |
| | | superfamily, | | | | | | | | | | | | | | |
| | | member 11a | | | | | | | | | | | | | | |
| 139 | RANTES | CCL5 | NP_002976.2 | 6352 | 2101 | 2101 | 2102 | 1875 | 1.12 | 2.1E−02 | 1.12 | 3.0E−02 | 1.12 | 2.8E−02 | −1.00 | 9.8E−01 |
| 140 | SCF | KIT ligand | NP_000890.1 | 4254 | 98 | 109 | 87 | 119 | −1.21 | 1.1E−01 | −1.09 | 5.1E−01 | −1.37 | 1.8E−02 | 1.26 | 5.3E−02 |
| 141 | SCFR | v-kit 4 feline | NP_000213.1 | 3815 | 5973 | 5453 | 6493 | 7516 | −1.26 | 1.2E−03 | −1.38 | 6.3E−05 | −1.16 | 9.7E−02 | −1.19 | 9.0E−02 |
| | | sarcoma viral | | | | | | | | | | | | | | |
| | | oncogene | | | | | | | | | | | | | | |
| 142 | SGP130 | neutrophil | | 4827 | 34497 | 34870 | 34123 | 47921 | −1.39 | 4.2E−05 | −1.37 | 8.7E−04 | −1.40 | 2.8E−04 | 1.02 | 8.4E−01 |
| | | migration | | | | | | | | | | | | | | |
| | | interleukin-1 | | | | | | | | | | | | | | |
| 143 | ST2 | receptor-like 1 | NP_003847.2 | 9173 | 673 | 743 | 602 | 750 | −1.11 | 3.9E−01 | −1.01 | 9.6E−01 | −1.24 | 1.2E−01 | 1.23 | 3.2E−01 |
| 144 | SURVIVIN | baculoviral | NP_001159.1 | 332 | 10889 | 9750 | 12028 | 13424 | −1.23 | 1.3E−01 | −1.38 | 3.1E−02 | −1.12 | 5.1E−01 | −1.23 | 2.3E−01 |
| | | IAP repeat- | | | | | | | | | | | | | | |
| | | containing 5 | | | | | | | | | | | | | | |
| 145 | TGFA | transforming | NP_003227.1 | 7039 | 168 | 172 | 165 | 221 | −1.31 | 1.6E−06 | −1.28 | 5.1E−04 | −1.34 | 5.5E−06 | 1.05 | 5.5E−01 |
| | | growth factor, | | | | | | | | | | | | | | |
| | | alpha | | | | | | | | | | | | | | |
| 146 | TIE2 | TEK tyrosine | NP_000450.1 | 7010 | 7410 | 7302 | 7518 | 9238 | −1.25 | 5.8E−02 | −1.27 | 7.2E−02 | −1.23 | 1.1E−01 | −1.03 | 8.2E−01 |
| | | kinase, | | | | | | | | | | | | | | |
| | | endothelial | | | | | | | | | | | | | | |
| | | tissue | | | | | | | | | | | | | | |
| 147 | TIMP1 | inhibitor of | NP_003245.1 | 7076 | 57178 | 60895 | 53461 | 43261 | 1.32 | 5.5E−04 | 1.41 | 1.1E−04 | 1.24 | 5.7E−02 | 1.14 | 1.8E−01 |
| | | metalloproteinase 1 | | | | | | | | | | | | | | |
| 148 | TNFA | TNF | NP_000585.2 | 7124 | 50 | 57 | 43 | 42 | 1.19 | 1.7E−01 | 1.35 | 6.6E−02 | 1.02 | 8.5E−01 | 1.32 | 6.3E−02 |
| | | superfamily, | | | | | | | | | | | | | | |
| | | member 2 | | | | | | | | | | | | | | |
| 149 | TNFB | TNF | NP_000586.2 | 4049 | 113 | 119 | 106 | 98 | 1.15 | 7.6E−03 | 1.22 | 3.4E−02 | 1.09 | 9.5E−02 | 1.12 | 2.3E−01 |
| | | superfamily, | | | | | | | | | | | | | | |
| | | member 1 | | | | | | | | | | | | | | |
| 150 | TNFR1 | TNFR | NP_001056.1 | 7132 | 268 | 319 | 217 | 217 | 1.23 | 2.3E−01 | 1.47 | 7.1E−02 | −1.00 | 1.0E+00 | 1.47 | 6.5E−02 |
| | | superfamily, | | | | | | | | | | | | | | |
| | | member 1A | | | | | | | | | | | | | | |
| 151 | TRAILR1 | TNFR | NP_003835.2 | 8797 | 98 | 103 | 93 | 139 | −1.43 | 3.0E−06 | −1.36 | 1.1E−03 | −1.49 | 3.7E−06 | 1.10 | 3.5E−01 |
| | | superfamily, | | | | | | | | | | | | | | |
| | | member 10a | | | | | | | | | | | | | | |
| 152 | TRAILR4 | TNFR | NP_003831.2 | 8793 | 3354 | 3719 | 2989 | 2658 | 1.26 | 3.0E−02 | 1.40 | 1.2E−02 | 1.12 | 3.6E−01 | 1.24 | 1.0E−01 |
| | | member 10d | | | | | | | | | | | | | | |
| 153 | TSH | thyroid | NP_000540.1 | 7252 | 375 | 398 | 353 | 416 | −1.11 | 2.2E−01 | −1.05 | 6.3E−01 | −1.18 | 7.6E−02 | 1.12 | 1.8E−01 |
| | | stimulating | | | | | | | | | | | | | | |
| | | hormone | | | | | | | | | | | | | | |
| 154 | UPA | plasminogen | NP_002649.1 | 5328 | 409 | 384 | 435 | 384 | 1.07 | 2.7E−01 | 1.00 | 9.9E−01 | 1.13 | 9.5E−02 | −1.13 | 1.5E−01 |
| | | activator, | | | | | | | | | | | | | | |
| | | urokinase | | | | | | | | | | | | | | |
| 155 | UPAR | plasminogen | NP_001005376.1 | 5329 | 4047 | 3833 | 4260 | 3729 | 1.09 | 1.7E−01 | 1.03 | 6.9E−01 | 1.14 | 7.8E−02 | −1.11 | 1.9E−01 |
| | | activator, | | | | | | | | | | | | | | |
| | | urokinase | | | | | | | | | | | | | | |
| | | receptor | | | | | | | | | | | | | | |
| 156 | VCAM1 | vascular cell | NP_001069.1 | 7412 | 43378 | 47459 | 39298 | 36307 | 1.19 | 1.2E−01 | 1.31 | 5.9E−02 | 1.08 | 5.1E−01 | 1.21 | 1.4E−01 |
| | | adhesion | | | | | | | | | | | | | | |
| | | molecule 1 | | | | | | | | | | | | | | |
| 157 | VECADHERIN | cadherin 5, | NP_001786.1 | 1003 | 31057 | 31538 | 30575 | 29957 | 1.04 | 5.3E−01 | 1.05 | 5.1E−01 | 1.02 | 7.7E−01 | 1.03 | 7.3E−01 |
| | | type 2, VE- | | | | | | | | | | | | | | |
| | | cadherin | | | | | | | | | | | | | | |
| 158 | VEGF | vascular | NP_003367.3 | 7422 | 421 | 494 | 347 | 357 | 1.18 | 2.2E−01 | 1.38 | 1.8E−01 | −1.03 | 5.3E−01 | 1.42 | 1.5E−01 |
| | | endothelial | | | | | | | | | | | | | | |
| | | growth factor | | | | | | | | | | | | | | |
| 159 | VEGFD | vascular | NP_004460.1 | 2277 | 7109 | 7107 | 7112 | 7608 | −1.07 | 2.7E−01 | −1.07 | 4.1E−01 | −1.07 | 3.3E−01 | −1.00 | 9.9E−01 |
| | | endothelial | | | | | | | | | | | | | | |
| | | growth factor D | | | | | | | | | | | | | | |
| 160 | VEGFR2 | kinase insert | NP_002244.1 | 3791 | 2714 | 2653 | 2775 | 2813 | −1.04 | 7.0E−01 | −1.06 | 6.0E−01 | −1.01 | 9.2E−01 | −1.05 | 7.7E−01 |
| | | domain | | | | | | | | | | | | | | |
| | | receptor | | | | | | | | | | | | | | |
| 161 | VEGFR3 | fms-related | NP_002011.1 | 2324 | 1461 | 1389 | 1533 | 1440 | 1.02 | 8.5E−01 | −1.04 | 6.7E−01 | 1.07 | 5.5E−01 | −1.10 | 3.6E−01 |
| | | tyrosine | | | | | | | | | | | | | | |
| | | kinase 4 | | | | | | | | | | | | | | |

^{a}pg/ml
^{b}IP-10 was measured by Luminex bead immunoassays

Of the 161 analytes measured, the serum levels of 30 (19%) analytes were significantly different in at least one inter-group comparison (mean fold change (FC)≧1.5 and p<0.05 by the Student's unpaired t-test). The data are presented in Table 3 as mean fold-change of the group comparisons. Serum levels of all 30 analytes differed between the IFN-hi SLE group and the control group. Serum levels of most analytes varied between the IFN-lo SLE group and the control group, or between all SLE cases and controls. The majority of analytes identified (23/30, 77%) exhibited higher serum levels in one or more of the SLE groups compared to controls. The levels of most CD4+ T helper-1 (TH1) cytokine polypeptides (e.g., IL-2, TNF-α, and IFN-γ) and TH2 cytokine polypeptides (e.g., IL-4, IL-9, IL-10, and IL-13) were similar in both the SLE and control groups (Table 2).

TABLE 3
Thirty polypeptide analytes dysregulated in SLE serum

| | All | | | |
|---|---|---|---|---|
| | SLE v Ctrl | SLE IFN-hi v | SLE IFN-lo v | SLE IFN-hi v |
| Analyte | FC | Ctrl FC | Ctrl FC | IFN-lo FC |
| IFN-ω | −1.88*** | −1.87*** | −1.89*** | 1.01 |
| ACE | −1.34** | −1.55*** | −1.18 | −1.32 |
| FGF RIII | −1.48** | −1.50* | −1.46* | −1.02 |
| ACE-2 | −1.09 | −1.46** | 1.15 | −1.68* |
| PDGF- | −1.50*** | −1.46** | −1.55*** | 1.06 |
| RA | | | | |
| CCL20 | −1.48** | −1.43* | −1.53** | 1.06 |
| (MIP-3A) | | | | |
| FGF-2 | −1.41*** | −1.34* | −1.50*** | 1.12 |
| GDNF | 1.43*** | 1.53** | 1.33* | 1.15 |
| BDNF | 1.21 | 1.55* | −1.14 | 1.76* |
| ICAM3 | 1.31** | 1.56** | 1.07 | 1.46* |
| CXCL11 | 1.23 | 1.58* | −1.15 | 1.81* |
| (I-TAC) | | | | |
| CCL7 | 1.57***** | 1.63*** | 1.51** | 1.08 |
| (MCP-3) | | | | |
| MMP7 | 1.58** | 1.64** | 1.53 | 1.07 |
| IL-18 | 1.46** | 1.71** | 1.22 | 1.40* |
| IL-5 | 1.54*** | 1.72** | 1.37* | 1.26 |
| CCL17 | 1.61* | 1.74* | 1.48 | 1.17 |
| (TARC) | | | | |
| IL-2SRA | 1.71**** | 1.81** | 1.61** | 1.12 |
| IL-15 | 1.58** | 1.88* | 1.28*** | 1.47 |
| CCL3 | 1.69***** | 2.00***** | 1.38** | 1.45*** |
| (MIP-1A) | | | | |
| CXCL2 | 1.82* | 2.13* | 1.52 | 1.40 |
| (GROB) | | | | |
| EGF | 2.12** | 2.16** | 2.08* | 1.04 |
| CXCL13 | 1.96*** | 2.18** | 1.73 | 1.26 |
| (BLC) | | | | |
| TGF-B | 2.18** | 2.34** | 2.02* | 1.15 |
| RIII | | | | |
| CXCL8 | 2.31**** | 2.49** | 2.14** | 1.16 |
| (IL-8) | | | | |
| CCL19 | 2.05*** | 2.82**** | 1.27 | 2.22*** |
| (MIP-3B) | | | | |
| IL-6 | 2.83***** | 3.11**** | 2.55** | 1.22 |
| CXCL9 | 2.29* | 3.24* | 1.34 | 2.42* |
| (MIG) | | | | |
| CCL8 | 2.48**** | 3.27*** | 1.69* | 1.93* |
| (MCP-2) | | | | |
| CCL2 | 3.78*** | 5.52** | 2.04 | 2.71* |
| (MCP-1) | | | | |
| CXCL10 | 5.82** | 9.09** | 1.74* | 5.82** |
| (IP-10) | | | | |

p < 0.05;
**p < 0.01;
***p < 0.001;
****p < 0.00001;
*****p < 0.000001
Analytes regulated by type I TFN are highlighted in bold font in Table 3.

Down-regulated analytes included IFN-ω, angiotensin converting enzyme (ACE), ACE-2, the chemotactic cytokine (chemokine) CCL20, the growth factor FGF-2, and soluble growth factor receptors (FGF R3 and PDGF-RA). Up-regulated analytes included several cytokine polypeptides (IL-5, IL-6, IL-15, IL-18, BDNF, and GDNF), the cytokine receptors IL-2SRA and TGF-B RIII, matrix metalloproteinase 7 (MMP7), and the adhesion molecule ICAM-3. Twelve of the 23 up-regulated analytes identified were chemokines, including representatives of both the CC- and CXC-families (Zlotnik and Yoshie, Immunity 12:121-127 (2000)).

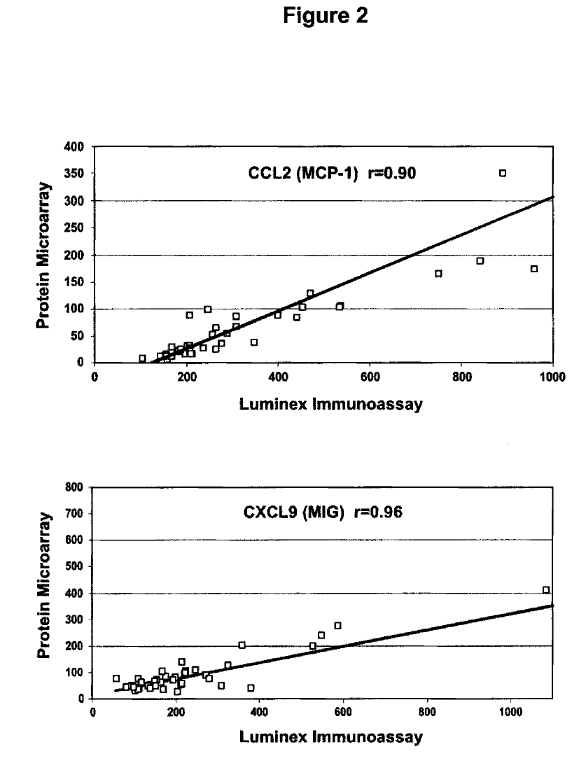

To validate a subset of the results obtained using the antibody array platform, the levels of two of the chemokines, CCL2 and CXCL9, were measured in 40 serum samples (15 IFN-hi, 12 IFN-lo, and 13 control samples) using Protein Multiplex Immunoassays (Biosource, Camarillo, Calif.) coupled with xMAP technology (Luminex, Austin, Tex.). Samples were analyzed in duplicate, and calibrated recombinant polypeptides were used to generate standard curves. The average coefficient of variance for duplicates was 10.7%. Linear regression was used to calculate correlation coefficients between results obtained using the antibody array platform and the Luminex assay. These analyses indicated that there was a high level of concordance between the two platforms (FIG. 2).

Concentration values for each analyte listed in Table 3 were normalized to the mean of the controls, log₂ transformed, subjected to unsupervised hierarchical clustering (CLUSTER), and visualized using TREEVIEW (Eisen et al., Proc Natl Acad Sci USA 95:14863-14868 (1998)). Hierarchical clustering analysis revealed that many of these polypeptides were coordinately dysregulated in the serum of a large percentage of IFN-hi patients and in the serum of a smaller fraction of IFN-lo patients.

To identify analytes that are regulated by type I IFN, gene expression experiments were performed in vitro. Peripheral blood mononuclear cells (PBMCs) from healthy donors were isolated using Lymphocyte Separation Medium (Mediatech Cellgro, Herndon, Va.). PBMCs (2 million cells/mL) were resuspended in complete medium (RPMI 1640, 2 mM L-glutamine, 50 units/mL penicillin, 50 μg/mL streptomycin) with 10% autologous plasma. Cells were incubated for 6 or 24 hours with medium alone, or with IFNα/IFNβ (1000 units/mL each; R&D Systems, Minneapolis, Minn.). Total RNA was isolated from the cells and converted to cRNA (Ambion, Austin, Tex.), which was hybridized to Affymetrix U133A gene expression arrays. The gene expression data were analyzed using Affymetrix Microarray Suite 5.0 software. Transcripts were considered to be regulated by IFN if their mean expression values changed by greater than 2-fold in cells stimulated with IFN for 6 or 24 hours compared to corresponding control cells that were not stimulated, and if the difference in expression value was greater than 500 Affymetrix units.

For the analytes listed in Table 3, gene expression data from the in vitro experiments, in which normal PBMCs were stimulated with type I IFN for 6 and 24 hours, were clustered as described above. The data were normalized to control conditions (i.e., incubation with medium-alone). The hierarchical clustering analysis identified analytes that were transcriptionally regulated by type I IFN, as determined by gene expression arrays. Analytes regulated by type I IFN are highlighted in bold font in Table 3. Most of the chemokines identified are inducible by type I IFN. Seven of the 11 analytes that differed in serum levels between IFN-hi and IFN-lo SLE cases were IFN-regulated chemokines.

These data indicate that serum levels of IFN-regulated chemokines are increased and serum polypeptide profiles are broadly dysregulated in many SLE patients. In addition, the highest levels of IFN-inducible analytes are found in patients carrying the IFN blood cell gene signature.

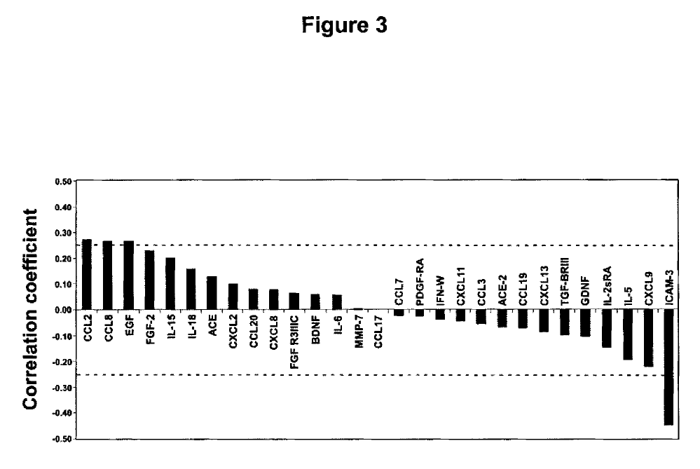

For the majority of individual analytes, there was a poor correlation between the level of gene expression observed in blood and the polypeptide level measured in serum (FIG. 3). In contrast, the IFN-regulated chemokine polypeptide levels were generally highly correlated with the presence of IFN-responsive gene transcripts in blood (UN gene score; Table 4). The IFN gene score was calculated based on expression of 82 IFN-inducible transcripts measured by concurrent whole blood gene expression microarrays, as described herein.

Clinical data, including disease activity indices (SLEDAI, SLAM-R) and laboratory results (anti-DNA antibodies (Abs), complement levels, erythrocyte sedimentation rates (ESR), white blood cell counts, hematocrit), were compared with analyte data using linear regression analysis (Pearson's correlation). One of the IFN-lo cases had insufficient clinical data and was excluded from the regression analyses. To determine the statistical significance of each comparison, random permutation analysis was performed to define the p-value thresholds.

Twenty analytes exhibited one or more significant correlations with clinical measures of disease activity (Table 4). Many of the IFN-regulated analytes exhibited strong positive correlations with the SLEDAI and the SLAM-R, two validated measures of global disease activity, as well as with the erythrocyte sedimentation rate (ESR) and titers of anti-dsDNA antibodies. The IFN-regulated analytes were negatively correlated with hematocrit (HCT) and complement C3 levels and exhibited similar negative trends with complement C4 levels and WBC counts.

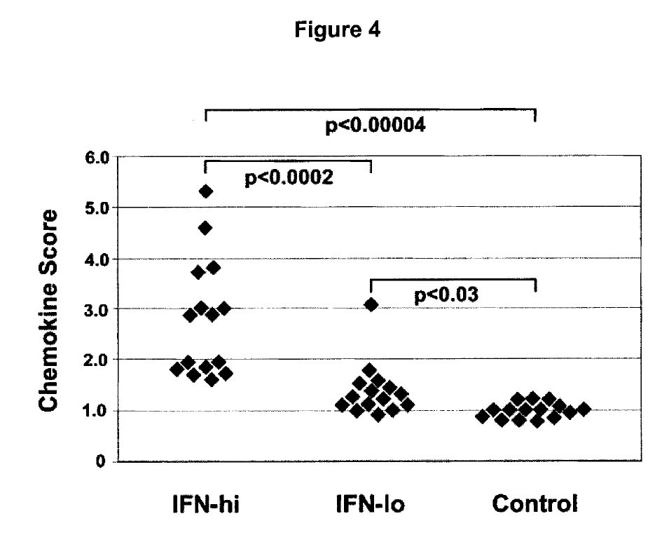

An IFN-regulated chemokine polypeptide score was derived from the normalized serum levels of seven IFN-regulated chemokines: CCL2 (MCP-1), CCL3 (MIP-1α), CCL8 (MCP-2), CCL19 (MIP-3β), CXCL9 (MIG), CXCL10 (IP-10), and CXCL11 (I-TAC). The concentration values were normalized across all samples so that the maximum value for any analyte was 1.0, and the values for each sample were summed to derive the final score (FIG. 4). The chemokine polypeptide score reflected the trends observed with the individual IFN-inducible analytes and exhibited stronger associations than any single analyte (Table 4). The chemokine polypeptide score was also more highly correlated with disease activity, as measured by SLEDAI, SLAM-R, ESR, and anti-DNA antibodies, than the IFN gene score (Table 4). These results indicate that there are striking clinical correlations between analytes, especially those that are IFN-regulated, and various measures of SLE disease activity.

TABLE 4
Association of IFN-regulated chemokines with SLE disease activity

| | IFN Gene | | | | Anti-DNA | | | | |
|---|---|---|---|---|---|---|---|---|---|
| Analyte | Score | SLEDAI | SLAM-R | ESR | Abs | HCT | C3 | C4 | WBC |
| ACE-2 | −0.41* | −0.24 | −0.20 | −0.08 | −0.25 | −0.28 | 0.28 | 0.23 | 0.07 |
| FGF R3 | −0.13 | −0.10 | 0.00 | 0.07 | −0.24 | 0.06 | 0.44* | 0.23 | 0.30 |
| MMP7 | 0.00 | 0.46* | 0.21 | 0.22 | 0.32 | −0.43** | −0.16 | 0.14 | −0.17 |
| TGF-B RIII | 0.04 | 0.38* | 0.17 | 0.17 | 0.45* | −0.05 | −0.06 | 0.05 | 0.05 |
| CCL17 (TARC) | 0.05 | 0.19 | 0.14 | −0.01 | 0.03 | 0.03 | 0.12 | 0.24 | 0.38* |
| GDNF | 0.12 | 0.45* | 0.44** | 0.44** | 0.39* | −0.31* | −0.13 | −0.19 | 0.01 |
| CXCL2 (GROB) | 0.12 | 0.39* | 0.30 | 0.15 | 0.22 | −0.15 | 0.15 | 0.29 | 0.17 |
| IL-5 | 0.17 | 0.40* | 0.40* | 0.24 | 0.35 | −0.31* | −0.09 | −0.07 | 0.08 |
| IL-6 | 0.18 | 0.26 | 0.37* | 0.40* | 0.27 | −0.32* | 0.01 | −0.13 | 0.11 |
| IL-15 | 0.20 | 0.63*** | 0.48** | 0.21 | 0.53** | −0.44** | −0.26 | −0.21 | −0.15 |
| IL-18 | 0.42* | 0.10 | 0.40* | 0.50** | 0.11 | −0.13 | −0.05 | 0.14 | 0.06 |
| BDNF | 0.45** | 0.12 | 0.05 | 0.00 | 0.12 | 0.14 | −0.29 | −0.15 | −0.03 |
| CCL8 (MCP-2) | 0.46** | 0.52** | 0.60*** | 0.62*** | 0.68*** | −0.52** | −0.43* | −0.32 | −0.30 |
| ICAM-3 | 0.48** | 0.38* | 0.55** | 0.54** | 0.20 | −0.62*** | −0.41* | −0.31 | −0.50** |
| CCL2 (MCP-1) | 0.48** | 0.35* | 0.39* | 0.43* | 0.57** | −0.21 | −0.40* | −0.33* | −0.19 |
| CXCL9 (MIG) | 0.54** | 0.17 | 0.48** | 0.56*** | −0.02 | −0.34* | −0.17 | −0.08 | −0.24 |
| CXCL11 (I-TAC) | 0.56*** | 0.42* | 0.67**** | 0.70**** | 0.60** | −0.56*** | −0.42* | −0.29 | −0.32 |
| CXCL10 (IP-10) | 0.58*** | 0.37* | 0.50** | 0.56*** | 0.48* | −0.23 | −0.40* | −0.33* | −0.31 |
| CCL19 (MIP-3B) | 0.59*** | 0.63*** | 0.57*** | 0.33* | 0.38* | −0.39* | −0.41* | −0.29 | −0.37* |
| CCL3 (MIP-1A) | 0.61*** | 0.59** | 0.68**** | 0.60*** | 0.61** | −0.56*** | −0.52** | −0.45* | −0.32 |
| Chemokine | 0.73**** | 0.57** | 0.72**** | 0.72**** | 0.61** | −0.46** | −0.49** | −0.37* | −0.32 |
| Protein Score | | | | | | | | | |
| IFN Gene Score | | 0.43* | 0.68**** | 0.52** | 0.35 | −0.26 | −0.51** | −0.39* | −0.40* |

*p < 0.05;
**p < 0.01;
***p < 0.001;
****p < 0.00001

Studies were then conducted to determine whether the chemokine levels measured in serum were correlated with specific organ system involvement. The patients were divided based on the presence or absence of organ system disease activity at the time of the visit (renal, serositis, hematologic, and skin), and levels of individual chemokines were compared (Tables 5 and 6). CXCL11 (I-TAC), CXCL13 (BLC), CXCL10 (IP-10) and CCL3 (MIP-1A) were present at significantly higher levels in serum of patients with active renal disease than those without. Levels of CCL8 (MCP-2), CCL2 (MCP-1) and CXCL2 (GROB) trended towards significance. The overall chemokine score was also elevated in patients with renal disease as compared to those without (Table 5).

Higher levels of CCL17 (TARC), CXCL10 (IP-10) and CCL2 (MCP-1) were found in the small subset of patients (N=4) with active serositis. A negative correlation was observed between levels of CCL20 (MIP-3A), CCL17 (TARC), CXCL2 (GROB) and CXCL8 (IL-8) and active hematologic system involvement (mostly thrombocytopenia; Table 5), and several additional chemokines (CXCL13, CCL3 and CXCL11) trended towards significance. No significant correlations were observed for patients with skin disease (Table 6). In a reciprocal approach, the 30 patients were ranked by serum levels of individual chemokines, and each group was divided evenly into chemokine ‘X’ high and low groups for comparison of clinical features. These data generally mirrored the findings shown in Table 5, demonstrating that many chemokines were associated with nephritis in this sample, and that lower chemokine levels were generally observed in individuals with hematologic involvement.

TABLE 5
Chemokine levels and clinical features of SLE

| | | | |
|---|---|---|---|
| | | Mean fold | |
| | | change | |
| Mean ± SD | Mean ± SD | (pos/neg) | P |

Renal

| | | | | |
|---|---|---|---|---|
| | Pos | Neg | | |
| | (N = 10) | (N = 20) | | |
| CXCL11 (I-TAC) | 288 ± 196 | 162 ± 67 | 1.77 | 0.014 |
| Chemokine score | 2.7 ± 1.5 | 1.7 ± 0.7 | 1.56 | 0.022 |
| CXCL13 (BLC) | 289 ± 191 | 169 ± 101 | 1.71 | 0.032 |
| CXCL10 (IP-10) | 127 ± 127 | 52 ± 59 | 2.43 | 0.045 |
| CCL3 (MIP-1A) | 429 ± 147 | 340 ± 87 | 1.26 | 0.047 |
| CCL8 (MCP-2) | 52 ± 37 | 32 ± 19 | 1.63 | 0.057 |
| CCL2 (MCP-1) | 136 ± 148 | 71 ± 56 | 1.92 | 0.090 |
| CXCL2 (GROB) | 1322 ± 933 | 742 ± 824 | 1.78 | 0.093 |

Serositis

| | | | | |
|---|---|---|---|---|
| | Pos | Neg | | |
| | (N = 4) | (N = 26) | | |
| CCL17 (TARC) | 128 ± 107 | 59 ± 40 | 2.16 | 0.019 |
| CXCL10 (IP-10) | 172 ± 179 | 61 ± 61 | 2.84 | 0.023 |
| CCL2 (MCP-1) | 175 ± 181 | 80 ± 79 | 2.21 | 0.070 |

Hematologic

| | | | | |
|---|---|---|---|---|
| | Pos | Neg | | |
| | (N = 7) | (N = 23) | | |
| CCL20 (MIP-3A) | 67 ± 9 | 83 ± 15 | 0.81 | 0.013 |
| CCL17 (TARC) | 26 ± 13 | 81 ± 58 | 0.33 | 0.020 |
| CXCL2 (GROB) | 332 ± 220 | 1119 ± 937 | 0.30 | 0.038 |
| CXCL8 (IL-8) | 5 ± 2 | 10 ± 6 | 0.50 | 0.038 |
| CXCL13 (BLC) | 120 ± 19 | 237 ± 157 | 0.51 | 0.062 |
| CCL3 (MIP-1A) | 302 ± 72 | 390 ± 120 | 0.77 | 0.076 |
| CXCL11 (I-TAC) | 128 ± 28 | 227 ± 147 | 0.56 | 0.089 |
| Chemokine score | 1.4 ± 0.4 | 2.3 ± 1.2 | 0.64 | 0.095 |

TABLE 6
Serum chemokine levels and specific SLE organ system involvement

| | | | | |
|---|---|---|---|---|
| AVG | SD | AVG | SD | P |

| | |
|---|---|
| Renal pos | Renal neg |
| (N = 10)^{a} | (N = 20) |

| | | | | | |
|---|---|---|---|---|---|
| CXCL13 (BLC)^{b} | 289 | 191 | 169 | 101 | 0.03 |
| CXCL2 (GROB) | 1322 | 933 | 742 | 824 | 0.1 |
| CXCL8 (IL-8) | 10 | 7 | 7 | 4 | 0.2 |
| CXCL10 (IP-10) | 127 | 127 | 52 | 59 | 0.05 |
| CXCL11 (I-TAC) | 288 | 196 | 162 | 67 | 0.01 |
| CCL2 (MCP-1) | 136 | 148 | 71 | 56 | 0.1 |
| CCL8 (MCP-2) | 52 | 37 | 32 | 19 | 0.1 |
| CCL7 (MCP-3) | 53 | 19 | 45 | 11 | 0.2 |
| CXCL9 (MIG) | 168 | 189 | 106 | 104 | 0.3 |
| CCL3 (MIP-1A) | 429 | 147 | 340 | 87 | 0.05 |
| CCL20 (MIP-3A) | 84 | 13 | 76 | 16 | 0.2 |
| CCL19 (MIP-3B) | 190 | 107 | 142 | 93 | 0.2 |
| CCL17 (TARC) | 71 | 43 | 67 | 62 | 0.8 |
| CK score (0-7) | 2.7 | 1.5 | 1.7 | 0.7 | 0.02 |

| | |
|---|---|
| Sero | Sero neg |
| pos (N = 4) | (N = 26) |

| | | | | | |
|---|---|---|---|---|---|
| CXCL13 (BLC) | 301 | 203 | 195 | 135 | 0.2 |
| CXCL2 (GROB) | 961 | 396 | 932 | 948 | 1.0 |
| CXCL8 (IL-8) | 12 | 10 | 8 | 4 | 0.1 |
| CXCL10 (IP-10) | 172 | 179 | 61 | 61 | 0.02 |
| CXCL11 (I-TAC) | 278 | 200 | 193 | 125 | 0.3 |
| CCL2 (MCP-1) | 175 | 181 | 80 | 79 | 0.1 |
| CCL8 (MCP-2) | 55 | 55 | 36 | 21 | 0.2 |
| CCL7 (MCP-3) | 43 | 9 | 49 | 15 | 0.5 |
| CXCL9 (MIG) | 127 | 75 | 127 | 147 | 1.0 |
| CCL3 (MIP-1A) | 445 | 55 | 358 | 119 | 0.2 |
| CCL20 (MIP-3A) | 79 | 19 | 79 | 15 | 1.0 |
| CCL19 (MIP-3B) | 143 | 92 | 161 | 101 | 0.7 |
| CCL17 (TARC) | 128 | 107 | 59 | 40 | 0.02 |
| CK score (0-7) | 2.8 | 1.8 | 1.9 | 1.0 | 0.2 |

| | |
|---|---|
| Heme pos | Heme neg |
| (N = 7) | (N = 23) |

| | | | | | |
|---|---|---|---|---|---|
| CXCL13 (BLC) | 120 | 19 | 237 | 157 | 0.1 |
| CXCL2 (GROB) | 332 | 220 | 1119 | 937 | 0.04 |
| CXCL8 (IL-8) | 5 | 2 | 10 | 6 | 0.04 |
| CXCL10 (IP-10) | 39 | 18 | 88 | 102 | 0.3 |
| CXCL11 (I-TAC) | 128 | 28 | 227 | 147 | 0.1 |
| CCL2 (MCP-1) | 39 | 25 | 109 | 107 | 0.1 |
| CCL8 (MCP-2) | 24 | 12 | 43 | 29 | 0.1 |
| CCL7 (MCP-3) | 41 | 12 | 50 | 14 | 0.1 |
| CXCL9 (MIG) | 81 | 84 | 141 | 150 | 0.3 |
| CCL3 (MIP-1A) | 302 | 72 | 390 | 120 | 0.1 |
| CCL20 (MIP-3A) | 67 | 9 | 83 | 15 | 0.01 |
| CCL19 (MIP-3B) | 145 | 83 | 162 | 104 | 0.7 |
| CCL17 (TARC) | 26 | 13 | 81 | 58 | 0.02 |
| CK score (0-7) | 1.4 | 0.4 | 2.3 | 1.2 | 0.1 |

| | |
|---|---|
| Skin pos | Skin neg |
| (N = 6) | (N = 24) |

| | | | | | |
|---|---|---|---|---|---|
| CXCL13 (BLC) | 162 | 43 | 221 | 160 | 0.4 |
| CXCL2 (GROB) | 1335 | 1281 | 836 | 768 | 0.2 |
| CXCL8 (IL-8) | 6 | 3 | 9 | 6 | 0.2 |
| CXCL10 (IP-10) | 90 | 84 | 73 | 96 | 0.7 |
| CXCL11 (I-TAC) | 178 | 63 | 211 | 149 | 0.6 |
| CCL2 (MCP-1) | 131 | 113 | 83 | 95 | 0.3 |
| CCLX (MCP-2) | 41 | 23 | 38 | 29 | 0.8 |
| CCL7 (MCP-3) | 43 | 10 | 49 | 15 | 0.4 |
| CXCL9 (MIG) | 105 | 75 | 133 | 151 | 0.7 |
| CCL3 (MIP-1A) | 360 | 102 | 372 | 121 | 0.8 |
| CCL20 (MIP-3A) | 86 | 15 | 77 | 15 | 0.2 |
| CCL19 (MIP-3B) | 187 | 145 | 151 | 86 | 0.4 |
| CCL17 (TARC) | 69 | 34 | 68 | 61 | 1.0 |
| CK score (0-7) | 2.2 | 0.9 | 2.0 | 1.2 | 0.8 |

^{a}For each chemokine and the chemokine score (CK score), an unpaired T-Test was used to determine differences in chemokine levels between patients positive (pos) or negative (neg) for renal, serositis, hematologic, and skin manifestations.
^{b}Serum chemokine levels reported in pg/ml.

The dataset was expanded to validate candidate SLE biomarkers, including interferon-regulated chemokines, in patient serum. Luminex bead-based technology was used to assay candidate SLE biomarkers in a group of 80 SLE patients with longitudinal visits (˜400 total samples). Serum was obtained and immediately treated with protease inhibitors. Longitudinal samples were available from most patients (average number of visits per patient ˜5), and all patients were evaluated by the same examining physician at every visit. Luminex immunoassays were used to quantitate 8 serum proteins identified as potential SLE biomarkers, as described above. As an initial analysis, a single visit from each patient was used to rank patients from highest to lowest concentration for each protein. Patients in the top and bottom quartiles (n=20 for each) were then compared for disease activity (SLEDAI, PGA, SLAM, BILAG) and other clinical features (DNA abs, ESR, complement, HCT, and WBC count) using unpaired Student's t-test.

Seven proteins showed significant (p<0.05) differences in SLE disease activity as measured by SLAM or SLEDAI, with the interferon-regulated chemokines MCP-2 (SLAM p<0.0006) and IP-10 (SLAM p<0.006, SLEDAI p<0.0002) exhibiting the most significant differences (Tables 7-10). Several laboratory measures including ESR, WBC count, low C3 and C4, and DNA abs were also significantly different between the groups (Table 7). Of particular interest were chemokines IP-10 (ESR p<0.000002, WBC count p<0.003), ENA-78 (ESR p<0.0001) and I-TAC (low C3 p<0.03, low C4 p<0.02, DNA abs p<0.02). Similar results were obtained when comparing protein levels between patient groups based on their ranking by clinical variables (Tables 8-10).

TABLE 7
Clinical feature p-values of the top and bottom 25% of patients as
ranked by analyte levels

| | Disease Activity | | | | | |
|---|---|---|---|---|---|---|
| | PGA | C: joints | B: renal | SLEDAI | BILAG | SLAM-R |
| TNF-RI | 0.38 | 0.92 | 0.01* | 0.25 | 0.06 | 0.01 |
| MCP-2 | 0.06 | 0.16 | 0.02 | 0.08 | 0.54 | 0.0006 |
| IP-10 | 0.53 | 0.63 | 0.76 | 0.0002 | 0.24 | 0.006 |
| MIP-1B | 0.98 | 0.31 | 0.64 | 0.06 | 0.91 | 0.02 |
| MMP-7 | 0.36 | 0.60 | 0.06 | 0.06 | 0.03 | 0.03 |
| MCP-1 | 0.38 | 0.11 | 0.44 | 0.15 | 0.56 | 0.61 |
| ENA-78 | 0.14 | 0.01 | 0.006 | 0.08 | 0.67 | 0.02 |
| I-TAC | 0.52 | 0.89 | 0.09 | 0.15 | 0.34 | 0.01 |

| | Lab Tests | | | | | | |
|---|---|---|---|---|---|---|---|
| | HCT | WBC | LYMPH | ESR | C3 | C4 | DNAP |
| TNF-RI | 0.86 | 0.30 | 0.88 | 0.01* | 0.92 | 0.67 | 0.66 |
| MCP-2 | 0.03 | 0.02 | 0.08 | 0.00002 | 0.22 | 0.18 | 0.41 |
| IP-10 | 0.001 | 0.003 | 0.02 | 0.000002 | 0.03 | 0.01 | 0.07 |
| MIP-1B | 0.16 | 0.06 | 0.29 | 0.03 | 0.60 | 0.64 | 0.03 |
| MMP-7 | 0.20 | 0.49 | 0.86 | 0.14 | 0.47 | 0.14 | 0.76 |
| MCP-1 | 0.32 | 0.93 | 0.01 | 0.93 | 0.02 | 0.12 | 0.02 |
| ENA-78 | 0.01 | 0.53 | 0.02 | 0.0001 | 0.01 | 0.04 | 0.28 |
| I-TAC | 0.0004 | 0.08 | 0.02 | 0.003 | 0.03 | 0.02 | 0.03 |

*Underlined values indicate p < 0.05

TABLE 8
Analyte level p-values of the top and bottom 25% of patients as
ranked by analyte levels

| | SLEDAI | SLAM | ESR |
|---|---|---|---|
| TNF-RI | 0.10 | 0.01* | 0.01 |
| MCP-2 | 0.07 | 0.02 | 0.0007 |
| IP-10 | 0.01 | 0.08 | 0.01 |
| MIP-1B | 0.71 | 0.74 | 0.30 |
| MMP-7 | 0.20 | 0.07 | 0.33 |
| MCP-1 | 0.09 | 0.11 | 0.34 |
| ENA-78 | 0.09 | 0.10 | 0.0015 |
| I-TAC | 0.10 | 0.02 | 0.0068 |

*Underlined values indicate p < 0.05

TABLE 9
Analyte level p-values in active vs. inactive patients

| | Definition 1 | Definition 2 |
|---|---|---|
| TNF-RI | 0.10 | 0.0061* |
| MCP-2 | 0.03 | 0.01 |
| IP-10 | 0.02 | 0.38 |
| MIP-1B | 0.16 | 0.20 |
| MMP-7 | 0.05 | 0.0091 |
| MCP-1 | 0.04 | 0.17 |
| ENA-78 | 0.23 | 0.25 |
| I-TAC | 0.07 | 0.03 |

*Underlined values indicate p < 0.05

TABLE 10
Average analyte level

| | SLEDAI | SLEDAI | Slam | Slam |
|---|---|---|---|---|
| | active | inactive | active | inactive |
| TNF-RI | 2846 | 1913 | 2897* | 1666 |
| MCP-2 | 62 | 35 | 62 | 35 |
| IP-10 | 260 | 53 | 202 | 126 |
| MIP-1B | 212 | 94 | 160 | 103 |
| MMP-7 | 12923 | 7529 | 12696 | 6254 |
| MCP-1 | 389 | 178 | 313 | 215 |
| ENA-78 | 4777 | 2703 | 3725 | 2188 |
| I-TAC | 505 | 223 | 573 | 204 |

*Underlined values indicate p < 0.05

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal having systemic lupus erythematosus, said method comprising (a) determining whether or not serum from a mammal comprises a signature selected from the group consisting of an activity signature 1, an activity signature 2, an activity signature 3, and an activity signature 4, and (b) classifying said mammal as having systemic lupus erythematosus if said serum comprises said signature and classifying said mammal as not having systemic lupus erythematosus if said serum does not comprise said signature.

2. The method of claim 1, wherein said mammal is a human.

3. The method of claim 1, wherein said method comprises determining whether or not said serum comprises said activity signature 1.

4. The method of claim 1, wherein said method comprises determining whether or not said serum comprises said activity signature 2.

5. The method of claim 1, wherein said method comprises determining whether or not said serum comprises said activity signature 3.

6. The method of claim 1, wherein said method comprises determining whether or not said serum comprises said activity signature 4.

7. A method for assessing systemic lupus erythematosus disease activity, said method comprising (a) determining whether or not serum from a mammal comprises a signature selected from the group consisting of an activity signature 1, an activity signature 2, an activity signature 3, and an activity signature 4, and (b) classifying said mammal as having active systemic lupus erythematosus disease if said serum comprises said signature and classifying said mammal as not having active systemic lupus erythematosus disease if said serum does not comprise said signature.

8. The method of claim 7, wherein said mammal is a human.

9. The method of claim 7, wherein said method comprises determining whether or not said serum comprises said activity signature 1.

10. The method of claim 7, wherein said method comprises determining whether or not said serum comprises said activity signature 2.

11. The method of claim 7, wherein said method comprises determining whether or not said serum comprises said activity signature 3.

12. The method of claim 7, wherein said method comprises determining whether or not said serum comprises said activity signature 4.

13. A method for determining whether or not a mammal is likely to experience active systemic lupus erythematosus disease, said method comprising (a) determining whether or not serum from a mammal comprises a signature selected from the group consisting of an activity signature 1, an activity signature 2, an activity signature 3, and an activity signature 4, and (b) classifying said mammal as being likely to experience said active systemic lupus erythematosus disease if said serum comprises said signature and classifying said mammal as not being likely to experience said active systemic lupus erythematosus disease if said serum does not comprise said signature.

14. The method of claim 13, wherein said mammal is a human.

15. The method of claim 13, wherein said method comprises determining whether or not said serum comprises said activity signature 1.

16. The method of claim 13, wherein said method comprises determining whether or not said serum comprises said activity signature 2.

17. The method of claim 13, wherein said method comprises determining whether or not said serum comprises said activity signature 3.

18. The method of claim 13, wherein said method comprises determining whether or not said serum comprises said activity signature 4.

19. A method for assessing effectiveness of a treatment for systemic lupus erythematosus disease, said method comprising determining whether or not a mammal having systemic lupus erythematosus disease and having received a treatment for said systemic erythematosus disease comprises serum comprising a signature selected from the group consisting of an activity signature 1, an activity signature 2, an activity signature 3, and an activity signature 4 to a level less than the level present in an earlier obtained serum sample from said mammal, wherein the presence of said serum indicates that said treatment is effective.

20. The method of claim 19, wherein said mammal is a human.

21. The method of claim 19, wherein said method comprises determining whether or not said serum comprises said activity signature 1.

22. The method of claim 19, wherein said method comprises determining whether or not said serum comprises said activity signature 2.

23. The method of claim 19, wherein said method comprises determining whether or not said serum comprises said activity signature 3.

24. The method of claim 19, wherein said method comprises determining whether or not said serum comprises said activity signature 4.
